(19) United States
(12) Patent Application Publication
(10) Pub. No.: US 2022/0347163 A1
(43) Pub. Date: Nov. 3, 2022

(54) COMPOSITIONS AND METHODS FOR PROVIDING CARDIOPROTECTIVE EFFECTS

(71) Applicants: CORNELL UNIVERSITY, Ithaca, NY (US); ALBERT EINSTEIN COLLEGE OF MEDICINE, Bronx, NY (US)

(72) Inventors: Lorraine J. GUDAS, New York, NY (US); Xiao-Han Tang, Staten Island, NY (US); Roberto Levi, New York, NY (US); Jessica Gambardella, Bronx, NY (US); Gaetano Santulli, New York, NY (US)

(73) Assignees: CORNELL UNIVERSITY, Ithaca, NY (US); ALBERT EINSTEIN COLLEGE OF MEDICINE, Bronx, NY (US)

(21) Appl. No.: 17/855,772

(22) Filed: Jun. 30, 2022

Related U.S. Application Data
(63) Continuation-in-part of application No. 17/542,852, filed on Dec. 6, 2021.
Application No. 17/542,852 is a continuation of application No. 16/008,946, filed on Jun. 14, 2018, now Pat. No. 11,191,755.
Application No. 16/008,946 is a continuation-in-part of application No. 15/112,159, filed on Jul. 15, 2016, now Pat. No. 11,160,769.
Application No. 15/112,159 is a continuation-in-part of application No. PCT/US14/12083, filed on Jan. 17, 2014.
(60) Provisional application No. 61/990,808, filed on May 9, 2014.

(51) Int. Cl. A61K 31/4436; A61K 31/426; A61P 9/10; A61P 9/06; A61K 31/192

(52) U.S. Cl. CPC A61K 31/4436; A61K 31/426; A61P 9/10; A61P 9/06; A61K 31/192

(57) ABSTRACT

The invention provides compositions and methods for providing a cardioprotective effect in a subject. Specifically, the invention relates to compositions and methods for treating cardiovascular ailments, such as compositions and methods for attenuating or ameliorating the development of cardiac contractile dysfunction, myocardial fibrosis or maladaptive remodeling in a subject having or having had a myocardial infarction (MI) or at risk of having a myocardial infarction by administering a retinoic acid receptor-beta (RARβ) agonist.

### CROSS-REFERENCE TO RELATED APPLICATIONS

[1] This is a continuation-in part application of U.S. application Ser. No. 17/542,852, filed Dec. 6, 2021, which is a continuation of U.S. application Ser. No. 16/008,946, filed Jun. 14, 2018 (now U.S. Pat. No. 11,191,755), which is a continuation-in part application of U.S. application Ser. No. 15/112,159, filed Jul. 15, 2016 (now U.S. Pat. No. 11,160,769), which is a National Phase Application of PCT International Application PCT/US15/11820, filed Jan. 16, 2015, which is a continuation-in-part of PCT International Application PCT/US14/12083, filed Jan. 17, 2014 and also claims priority to and the benefit of U.S. Provisional Application 61/990,808, filed May 9, 2014. All applications above are incorporated by reference herein in their entirety.

### GOVERNMENT FUNDING

[2] This invention was made with Government support under Grant Number DK113088, awarded by the National Institutes of Health. The United States Government has certain rights in the invention.

### FIELD OF THE INVENTION

[3] The invention relates to compositions and methods for providing a cardioprotective effect in a subject. Specifically, the invention relates to compositions and methods for treating cardiovascular ailments, as well as for attenuating or ameliorating the development of cardiac contractile dysfunction, myocardial fibrosis or maladaptive remodeling in a subject having or having had a myocardial infarction (MI) or at risk of having a myocardial infarction by administering a retinoic acid receptor-beta (RARβ) agonist.

### BACKGROUND OF THE INVENTION

[4] Early reperfusion of an occluded coronary artery after myocardial infarction can reduce infarct size, which contributes to preserving left ventricular contraction and preventing the onset of heart failure and other pathologies. However, reperfusion paradoxically can cause further cardiomyocyte death, which is generally known as myocardial ischemia/reperfusion (I/R) injury. Ischemic reperfusion injury is accompanied by severe arrhythmias, such as ventricular tachycardia (VT) and agonist (VF), which are significant causes of sudden death in patients with ischemic heart disease. In addition, myocardial fibrosis is also a key feature of heart failure (HF), contributing significantly to contractile dysfunction.

[5] Vitamin A (retinol) and its metabolites and derivatives, collectively known as retinoids, are important lipophilic signaling molecules that play critical roles in controlling both vertebrate development and stem cell differentiation in the adult. The actions of retinoids, such as the potent, biologically active endogenous metabolite of vitamin A, all-trans retinoic acid (RA), are primarily mediated by binding to ligand-activated transcription factors, the retinoic acid receptors (RARs) α, β, and γ. When RARs bind the pan-agonist RA, they heterodimerize with retinoid X receptors (RXRs) α, β, and γ. Agonists that bind to different RAR receptors can be expected to have different effects because different RARs are known to sere different biological functions. The RARs are expressed in the heart during development and in the adult, and RA signaling is active in the post-ischemic heart. The RA signaling pathway can reduce cardiac I/R injury and ROS production. RA also displayed protective effects against cardiac arrhythmias. Moreover, all-trans retinoic acid protected against doxorubicin-induced cardiotoxicity, in which oxidative stress and UR-like damage are known to play a major role. Furthermore, supplementation with RA has been shown to prevent left ventricular (LV) dilatation and preserved ventricular function in rats with induced infarction. Conversely, in adult rats, vitamin A deficiency has been demonstrated to cause left ventricular dilatation that led to a major decrease in cardiac function. Loss of RARα specifically in mouse cardiomyocytes resulted in diastolic dysfunction from increased ROS.

[6] Accordingly, there remains an unmet need for therapeutic compositions and treatment methods capable of preventing myocardial reperfusion injury and reducing myocardial infarct (MI) size, so as to preserve heart function (LV systolic function) and prevent the onset of heart failure in patients presenting with myocardial infarction at risk of myocardial ischemia/reperfusion (UR) injury. There also remains an unmet need for therapeutic compositions and treatment methods that ameliorates myocardial fibrosis, attenuates the development of contractile dysfunction, and/or maladaptive remodeling in heart failure (HF).

### SUMMARY OF THE INVENTION

[7] In one aspect, the invention provides method of attenuating or ameliorating cardiac contractile dysfunction in a subject having or having had a myocardial infarction MI or at risk of having a myocardial infarction, comprising administering to the subject a therapeutically effective amount of a selective retinoic acid receptor-β₂ (RARβ₂) agonist or a pharmaceutically acceptable salt, ester, amide, prodrug thereof, or a combination thereof. In an exemplary embodiment, said RARβ agonist is AC261066.

[8] In another aspect, the invention provides a method of attenuating or ameliorating cardiac fibrosis in a subject having or having had a myocardial infarction (MI) or at risk of having a myocardial infarction, comprising administering to the subject a therapeutically effective amount of a selective retinoic acid receptor-β₂ (RARβ₂) agonist or a pharmaceutically acceptable salt, ester, amide, prodrug thereof, or a combination thereof.

[9] In an additional aspect, the invention provides a method of attenuating or ameliorating ventricular remodeling in a subject having or having had a myocardial infarction (MI) or at risk of having a myocardial infarction, comprising administering to the subject a therapeutically effective amount of a selective retinoic acid receptor-β₂ (RARβ₂) agonist or a pharmaceutically acceptable salt, ester, amide, prodrug thereof, or a combination thereof.

[10] In a further aspect, the invention provides a pharmaceutical formulation comprising a therapeutically effective amount of a retinoic acid receptor-beta (RARβ) agonist and a pharmaceutically acceptable excipient.

[11] Other features and advantages of this invention will become apparent from the following detailed description examples and figures. It should be understood, however, that the detailed description and the specific examples, while indicating various embodiments of the invention, are given by way of illustration only, since various changes and modifications within the spirit and scope of the invention will become apparent to those skilled in the art from the detailed description.

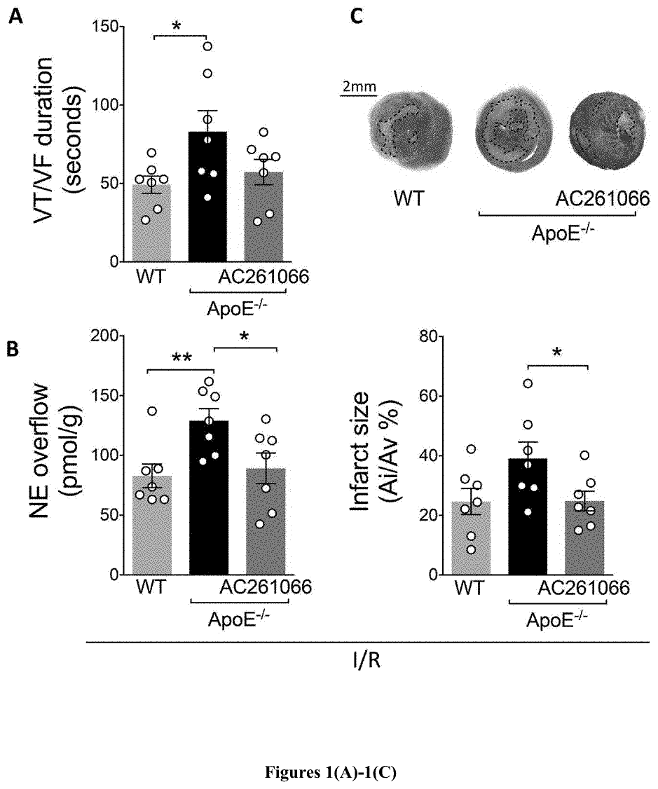

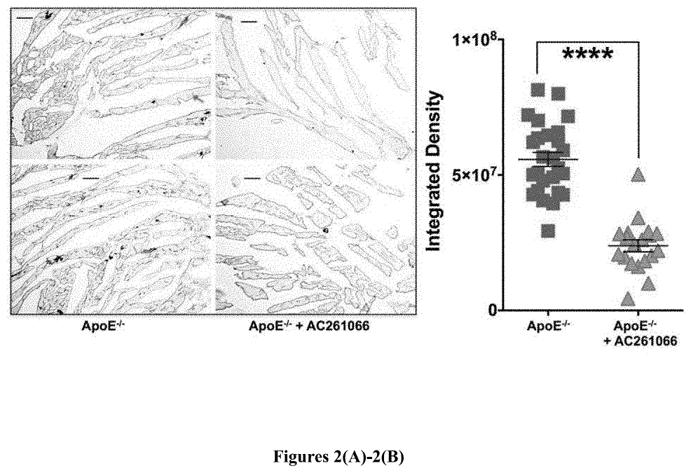

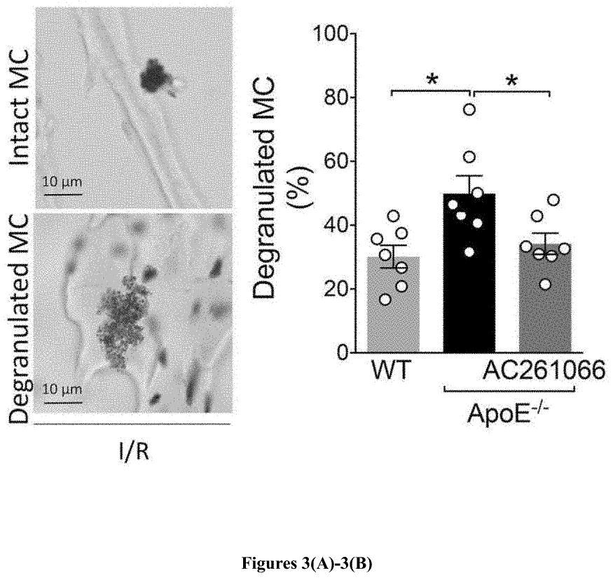

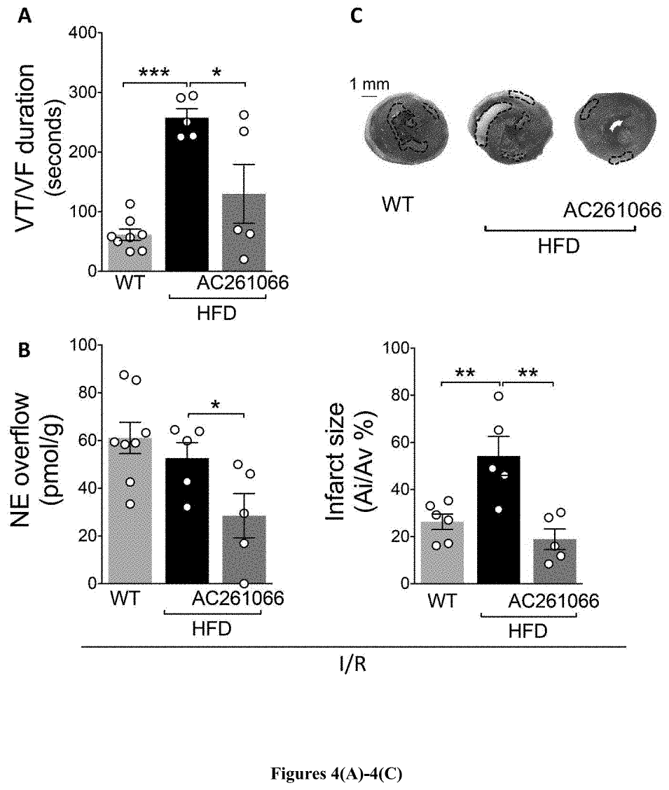

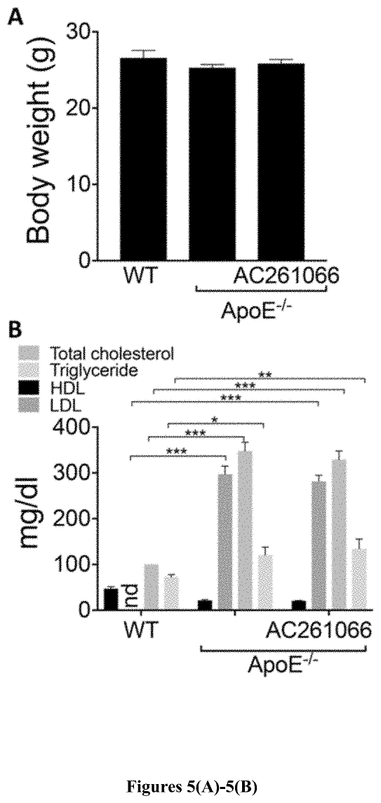

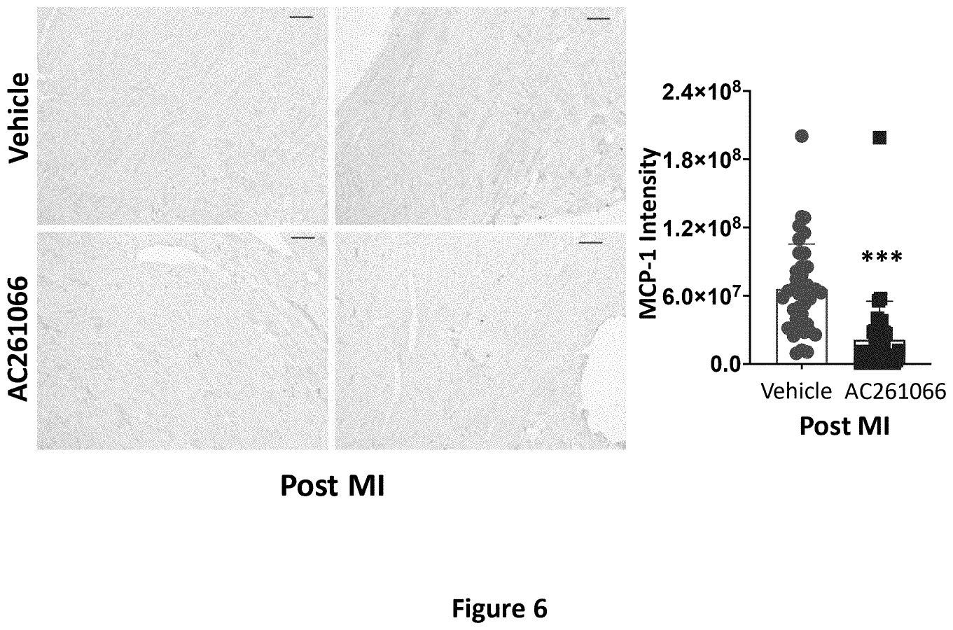

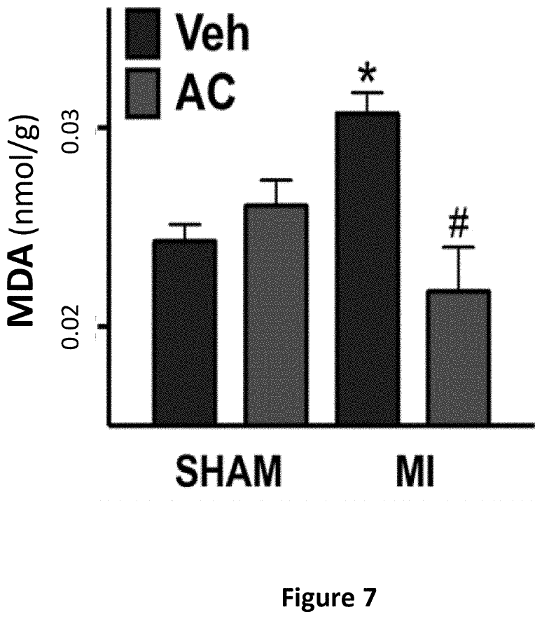

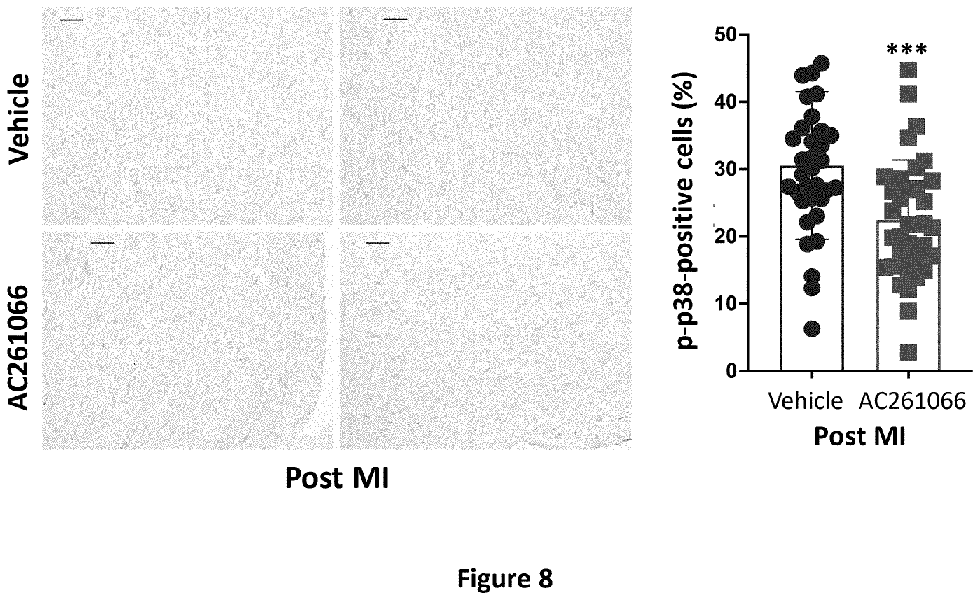

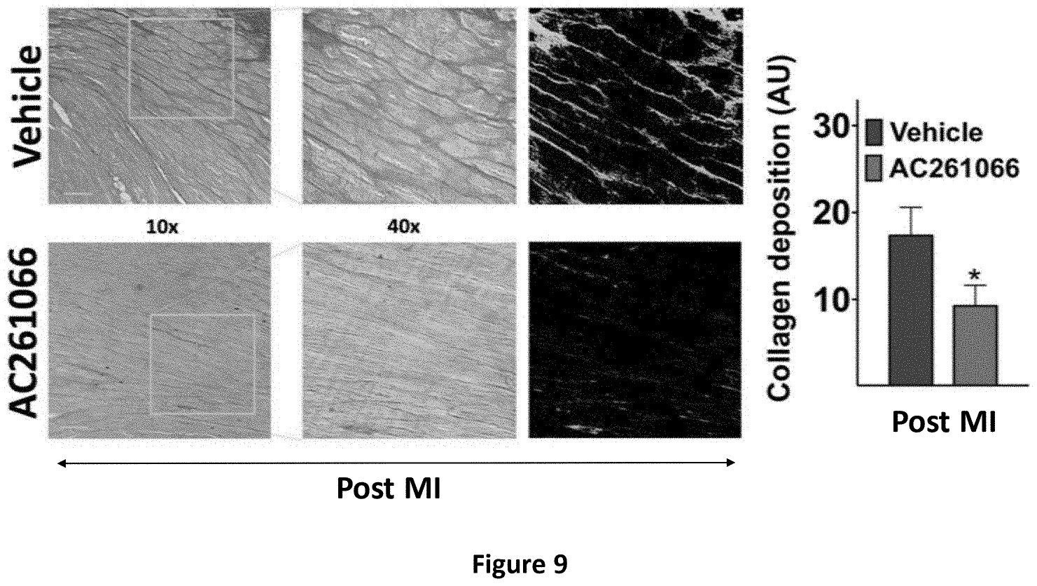

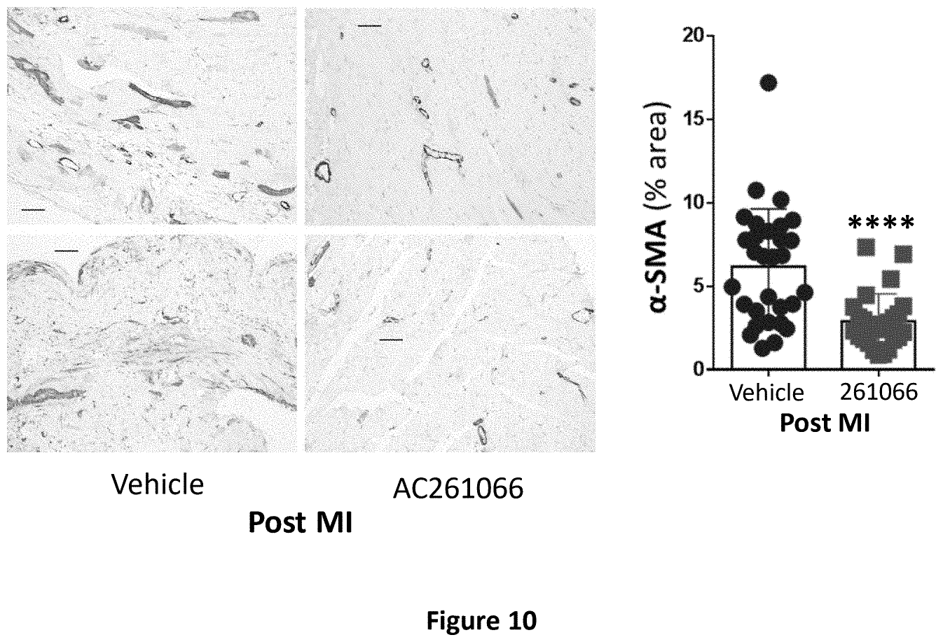

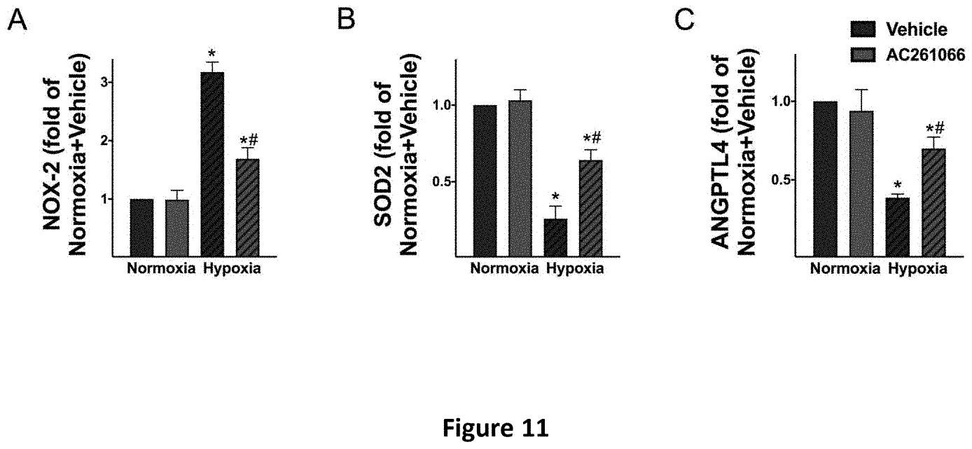

### BRIEF DESCRIPTION OF THE DRAWINGS

[12] FIGS. 1(A)-1(C) illustrate that the RARβ₂ agonist AC261066 reduces NE (norepinephrin) overflows, alleviates reperfusion arrhythmias, and decreases infarct sizes in hearts isolated from ApoE (apolipoprotein E)^{-/-} mice subjected to ex-vivo I/R. Mouse hearts were subjected to 40-minute global ischemia followed by 120-minute reperfusion (WT, n=7; ApoE^{-/-}, n=7, ApoE^{-/-}+AC261066, n=7). FIG. 1(A) graphically shows duration of reperfusion arrhythmias (VT/VF) in WT, ApoE^{-/-}and ApoE^{-/-}+AC261066. FIG. 1(B) graphically shows overflows of NE in the first 6 minutes from the start of reperfusion in WT, untreated or treated with AC261066 (ApoE^{-/-}and ApoE^{-/-}+AC261066, respectively). FIG. 1(C) shows a qualitative and quantitative representation of (2,3,5-Triphenyltetrazolium chloride (TTC) staining in 2-mm-thick left ventricle slices collected at the end of reperfusion. Bars indicate infarcted slice areas as a percentage of total slice areas (Ai/Av %; means ±SEM of independent experiments). Pale areas indicate I/R-injured tissue, while healthy tissue is colored in red. *, P <0.05; **, P <0.01, by unpaired t-test. Each open circle is one mouse.

[13] FIGS. 2(A)-2(B) illustrates malondialdehyde (MDA) levels in heart sections of ApoE^{-/-} mice, either untreated or treated with AC261066, subjected to ex-vivo I/R. Following I/R, hearts were fixed, embedded in optimal cutting temperature (OCT) compound, and sectioned. Sections were stained with an MDA antibody (Magnification: 200x), red arrow marks MDA signal. FIG. 2(A) shows representative images of oxidative stress staining in ApoE^{-/-}and ApoE^{-/-}+AC261066. Anti-malondialdehyde (MDA) stain in mouse heart frozen sections, the red arrow points to brown spots denoting MDA. Magnification: 200×, Scale bars: 50 gm. Two samples per group are shown. FIG. 2(B) is a graphic quantification of MDA levels in all fields. Six-eight representative areas of each heart section from 3-4 mice per group were photographed and analyzed. The quantification was carried out using ImageJ (NIH). Student's t test was used for statistical analysis (****P <0.0001).

[14] FIGS. 3(A)-3(B) show that the RARβ₂ agonist AC261066 reduces mast cell degranulation in ApoE^{-/-}mouse hearts subjected to ex-vivo I/R. Frozen heart sections of WT, ApoE^{-/-}and ApoE^{-/-}+AC2610166 mouse hearts subjected to I/R (n=7, 7, 7) were stained with toluidine blue. FIG. 3(A) shows representative images of intact and degranulated cardiac mast cells (MC) in WT hearts subjected to ex-vivo I/R. FIG. 3(B) is a graphic quantification of MC degranulation in WT, ApoE^{-/-}and ApoE^{-/-}+AC261066, calculated as a percentage of degranulated MC over total MC. *, P <0.05 by one-way ANOVA followed by Tukey's post-hoc test.

[15] FIGS. 4(A)-4(C) illustrate that the RARβ₂ agonist AC261066 reduces NE overflows, alleviates reperfusion arrhythmias and decreases infarct sizes in hearts from high fat diet (HFD)-fed wild type (WT) mice subjected to ex-vivo I/R. Mouse hearts were subjected to 40-minute global ischemia followed by 120-minute reperfusion (WT, n=8; HFD, n=5, HFD +AC261066, n=5). FIG. 4(A) shows duration of reperfusion arrhythmias (VT/VF) in WT, HFD and HFD +AC261066. FIG. 4(B) shows overflows of NE collected during 6 minutes from the start of reperfusion. FIG. 4(C) shows a qualitative and quantitative representation of TTC staining in 2-mm-thick left ventricle slices. Scale bar is 1 mm. Bars indicate means ±SEM of independent experiments. Pale areas indicate I/R-injured tissue, while healthy tissue is colored in red. *, P<0.05; **, P <0.01, by unpaired Student's t-test.

[16] FIGS. 5(A)-5(B) graphically illustrate that body weight and total cholesterol levels of ApoE^{-/-}mice are not affected by the RARβ₂ agonist AC261066. FIG. 5(A) shows body weight of WT, ApoE^{-/-}and AC261066-treated ApoE^{-/-}mice at the time of the experiment (WT, n=7; ApoE^{-/-}n=7, ApoE^{-/-}+AC261066, n=7). FIG. 5(B) shows blood levels of total cholesterol (light blue), triglycerides (yellow), HDL (black) and LDL (red; nd =not detectable in WT) in ApoE^{-/-}and AC261066-treated ApoE^{-/-}mice. Bars indicate means ±SEM. *, P <0.05; **, P <0.01; ***, P <0.001 by one-way ANOVA followed by Tukey's post-hoc test.

[17] FIG. 6 shows that the RARβ₂ agonist AC261066 reduces the MCP-1 protein (monocyte chemotactic protein-1) level in the hearts after permanent LAD (left anterior descending artery) occlusion. Four weeks after LAD occlusion, mouse hearts were fixed, embedded in paraffin, sectioned, and stained with an MCP-1 antibody. Representative pictures from five mice per group. Magnification, 200x; bar, 50 μm. The stained images (7-8 images per mouse) were quantified using FIJI software. Data are means ±S.D. ***P <0.001 (intensity).

[18] FIG. 7 shows that the RARβ₂ agonist AC261066 limits the increase in oxidative stress induced by MI in mouse hearts. Four weeks after LAD occlusion, MDA was measured in cardiac tissue with a Lipid Peroxidation Assay Kit (Abcam, ab118970). Data are means ±S.E.M. of triplicate experiments. *P <0.05 versus sham; #P <0.05 versus vehicle.

[19] FIG. 8 shows that the RARβ₂ agonist AC261066 reduces the phospho-p38 (p-p38) protein level in the hearts after permanent LAD occlusion. Four weeks after LAD occlusion, mouse hearts were fixed, embedded in paraffin, sectioned, and stained with a phospho-p38 (p-p38) antibody. Representative pictures from five mice per group. Magnification, 200×; bar, 50 μm. The stained images (seven images per mouse) were quantified using FIJI software. Data are means ±S.D. **P <0.01.

[20] FIG. 9 shows that the RARβ₂ agonist AC261066 limits cardiac fibrosis in mice after permanent LAD coronary artery occlusion. Four weeks after LAD occlusion, mouse hearts were fixed, embedded in paraffin, sectioned, and stained (Picrosirius red). Representative pictures from five mice per group. Bar, 100 μm. Collagen deposition was quantified using FIJI software (plugin commands: Edit>Invert) to obtain images in which Picrosirius red—positive fibers appear in light blue (panels on the right). Images were captured with a Zeiss Axio Z1 inverted microscope. Data are means ±S.D. *P <0.05.

[21] FIG. 10 shows that the RARβ₂ agonist AC261066 reduces the α-SMA (a smooth muscle action) protein level in the hearts after permanent LAD coronary artery occlusion. Four weeks after LAD occlusion, mouse hearts were fixed, embedded in paraffin, sectioned, and stained with an α-SMA antibody. Representative pictures from five mice per group. Magnification, 200x; bar, 50 μm. α-SMA stained images (6-7 images per mouse) were quantified using FIJI software. Data are means ±S.D. ****P <0.0001 (percent area).

[22] FIG. 11 shows that the RARβ₂ agonist AC261066 decreases the mRNA levels of one pro-oxidant gene (NOX-2) and increases the mRNA levels of two protective genes (SOD2, ANGPTL4) in cultured hypoxic murine cardiac fibroblasts. mRNA levels of the indicated genes were assessed by RTqPCR in murine cardiac fibroblasts in normoxic or hypoxic conditions for 12 hours and treated with AC261066 (100 nM, 12 hours) or vehicle. (3-actin was used as a normalizing mRNA. Data are means ±S.E.M. of triplicate experiments. *P <0.05 versus normoxia, #P <0.05 versus vehicle.

### DETAILED DESCRIPTION OF THE INVENTION

[23] The present subject matter may be understood more readily by reference to the following detailed description which forms a part of this disclosure. It is to be understood that this invention is not limited to the specific methods, products, conditions or parameters described and/or shown herein, and that the terminology used herein is for the purpose of describing particular embodiments by way of example only and is not intended to be limiting of the claimed invention.

[24] Unless otherwise defined herein, scientific and technical terms used in connection with the present application shall have the meanings that are commonly understood by those of ordinary skill in the art. Further, unless otherwise required by context, singular terms shall include pluralities and plural terms shall include the singular.

[25] As employed above and throughout the disclosure, the following terms and abbreviations, unless otherwise indicated, shall be understood to have the following meanings.

[26] In the present disclosure the singular forms “a,” “an,” and “the” include the plural reference, and reference to a particular numerical value includes at least that particular value, unless the context clearly indicates otherwise. Thus, for example, a reference to “a compound” is a reference to one or more of such compounds and equivalents thereof known to those skilled in the art, and so forth. The term “plurality”, as used herein, means more than one. When a range of values is expressed, another embodiment includes from the one particular and/or to the other particular value. Similarly, when values are expressed as approximations, by use of the antecedent “about,” it is understood that the particular value forms another embodiment. All ranges are inclusive and combinable.

[27] In the context of the present disclosure, by “about” a certain amount it is meant that the amount is within ±20% of the stated amount, or preferably within ±10% of the stated amount, or more preferably within ±5% of the stated amount.

[28] As used herein, the terms “component,” “composition,” “composition of compounds,” “compound,” “drug,” “pharmacologically active agent,” “active agent,” “therapeutic,” “therapy,” “treatment,” or “medicament” are used interchangeably herein to refer to a compound or compounds or composition of matter which, when administered to a subject (human or animal) induces a desired pharmacological and/or physiologic effect by local and/or systemic action.

[29] As used herein, the terms “treatment” or “therapy” (as well as different forms thereof) include preventative (e.g., prophylactic), curative or palliative treatment. As used herein, the term “treating” includes alleviating or reducing at least one adverse or negative effect or symptom of a condition, disease or disorder.

[30] The term “ischemia” refers herein to an inadequate blood supply, including stopped blood flow, to heart muscle, that prevents the heart muscle tissue from receiving enough oxygen to keep it alive. The terms “ischemia-reperfusion injury”, “reperfusion injury” and “reoxygenation injury” refer herein to tissue damage caused when the blood supply is returned to the tissue after a period of ischemia. The lack of oxygen and nutrients during ischemia creates a condition in which the restoration of circulation results in inflammation and oxidative damage through the induction of oxidative stress rather than (or along with) restoration of normal function.

[31] The terms “subject,” “individual,” and “patient” are used interchangeably herein, and refer to an animal, for example a human, to whom treatment, including prophylactic treatment, with the pharmaceutical composition according to the present invention, is provided. The term “subject” as used herein refers to human and non-human animals. The terms “non-human animals” and “non-human mammals” are used interchangeably herein and include all vertebrates, e.g., mammals, such as non-human primates, (particularly higher primates), sheep, dog, rodent, (e.g. mouse or rat), guinea pig, goat, pig, cat, rabbits, cows, horses and non-mammals such as reptiles, amphibians, chickens, and turkeys.

[32] As used herein, the terms “cardioprotective” effect and “cardioprotection” refer to and encompass methods of preserving the function and viability of cardiac muscle cells subjected to ischemia or to reperfusion injury, also called “ischemia-reperfusion injury” or “reoxygenation injury”. Cardioprotection may be implemented before an ischemic event (preconditioning), during an ischemic event (preconditioning) and after the event and during reperfusion (postconditioning). Cardioprotective methods provided herein performed prior to an ischemic event may provide immediate onset of protection against myocardial infarction up to at least six weeks or longer against myocardial infarction. In various embodiments, administration of a retinoic acid receptor-beta (RARβ) agonist to a subject in need thereof, in particular administration of a RARβ₂ agonist produces or exerts a cardioprotective effect, at a time period ranging from about 6 weeks to at least about one year, prior to a myocardial infarction in the subject.

[33] Cardioprotection may be affected during an ischemic event to provide immediate onset of protection against myocardial infarction by attenuating an infarct size in the heart, diminishing coronary norepinephrine spillover during reperfusion of the heart, reducing oxidative damage, and/or alleviating reperfusion arrhythmias. The reperfusion arrhythmias comprise ventricular tachycardia and/or ventricular fibrillation. The ventricular tachycardia and/or ventricular fibrillation (VF) may be present when the subject has a heart disease, i.e., prior to a myocardial infarction in the subject.

[34] The present invention relates to producing or providing a cardioprotective effect on a heart of a subject in need thereof, by administering to the subject a therapeutically effective amount of a retinoic acid receptor-beta (RARβ) agonist, in particular an RARβ₂ agonist. The present invention also relates to treatment methods for reducing a myocardial injury, alleviating arrhythmias associated with or present during myocardial infarction, as well as treating cardiac ailments, including but not limited to chronic arrhythmia and cardiac failure in a subject in need thereof. The present invention also relates to methods of attenuating or ameliorating cardiac contractile dysfunction, cardiac fibrosis and ventricular remodeling in a subject having or having had a myocardial infarction (MI) or at risk of having a myocardial infarction.

[35] While not intending to be bound by any particular mechanism of operation, it is hypothesized that the retinoic acid receptor-beta (RARβ) agonist, specifically an RARβ₂ agonist, administered according to the present invention effects or exerts cardioprotection by a reduction in oxidative stress and mast cell (MC) degranulation. Furthermore, it is commonly believed that the reduction in myocardial injury and adrenergic activation, and the antiarrhythmic effects result from decreased formation of oxygen radicals and toxic aldehydes; oxygen radicals and toxic aldehydes are known to elicit the release of MC-derived renin, promoting the activation of local renin-angiotensin system (RAS) leading to enhanced NE release and reperfusion arrhythmias. Furthermore, it is believed that the retinoic acid receptor-beta (RARβ) agonist, specifically an RARβ₂ agonist, administered according to the present invention ultimately alleviates post-MI cardiac dysfunction by sequentially activating RARβ₂ in cardiomyocytes and fibroblasts, and then regulating a set of genes responsible for decreasing oxidative stress and ROS production. These responsive genes then diminish interstitial fibrogenesis and remodeling.

[36] Various embodiments of the invention provide methods for producing cardioprotective effects (cardioprotection) and pharmaceutical formulations that comprise a retinoic acid receptor-beta (RARβ) agonist, more specifically, an RARβ₂ agonist. Such pharmaceutical formulations can be configured in various ways and in a variety of dosage forms, such as formulations for oral and peritoneal administration, to reduce oxidative stress and mast cell (MC) degranulation and/or to diminish coronary norepinephrine spillover during reperfusion of the heart.

[37] As used herein, the terms “decrease”, “reduce” and “diminish” are used interchangeably to refer to a negative change in the level, activity or function of a molecule, cell or organ. It is meant that the particular level, activity or function is lower by about 10%, about 25%, about 50%, about 75%, about 90%, about 1-fold, about 2-fold, about 5 fold, about 10-fold, about 25-fold, about 50-fold, or about 100 fold, or lower, when compared to a control.

[38] As used herein, the terms “elevate”, “increase”, “improve” and “enhance” are used interchangeably to refer to a positive change in the level, activity or function of a molecule, cell or organ. It is meant that the particular level, activity or function is higher by about 10%, about 25%, about 50%, about 75%, about 90%, about 1-fold, about 2-fold, about 5 fold, about 10-fold, about 25-fold, about 50-fold, or about 100 fold, or higher, when compared to a control.

[39] The retinoic acid receptor (RAR) is a type of nuclear receptor that is activated by both all-trans retinoic acid (RA). There are three retinoic acid receptors (RAR), RARα, RARβ, and RARγ, encoded by the RARα, RARβ, RARγ genes, respectively. Each receptor isoform has several splice variants: four- for α, four- for β, and two- for γ.

[40] RAR heterodimerizes with RXR and in the absence of ligand, the RAR/RXR dimer binds to hormone response elements known as retinoic acid response elements (RAREs) complexed with corepressor protein. Binding of agonist ligands to RAR results in dissociation of corepressor and recruitment of coactivator protein that, in turn, promotes transcription of the downstream target gene into mRNA and eventually protein.

[41] The RARβ subtype consists of four known isoforms RARβ₁, RARβ₂, RARβ₃ and RARβ₄. The ligand binding domains of the four isoforms are similar, while the variation between the isoforms is located within the proximal N-terminus, which encompasses the ligand-independent activation domain (AF-1).

[42] It has been reported that the ligand binding domain, i.e., AF-2, of a given RAR isotype cooperates with the AF-1 domain in a promoter context manner. The AF-2 domains are conserved between the isoforms, the AF-1 domains are not. Relying on RAR (e.g., RARβ₂) receptor-ligand crystal structure, various RAR agonists have been designed and identified.

[43] RARβ agonists include, but are not limited to, AC261066, AC55649, Tazarotene, Adapalene, 9-cis-retinoic acid and TTNPB. AC261066 and AC55649 are highly selective RARβ agonists. “Highly selective RARβ agonists” also include other agonists with a similar binding affinity as AC261066 or AC55649, e.g., at least 50% or greater, preferably 75% or greater, more preferably 90% or greater of the RARβ binding affinity of AC261066 or AC55649. “Highly-selective RARβ₂ agonists” include agonists with a similar binding affinity as AC261066 or AC55649, e.g., at least 50% or greater, preferably 75% or greater, more preferably 90% or greater of the RARβ₂ binding affinity of AC261066 or AC55649. A highly selective RARβ (e.g., RARβ₂) agonist preferably has an affinity for RARβ (e.g., RARβ₂) greater than 6.00 pEC₅₀, more preferably greater than 6.50 pEC₅₀, more preferably greater than 7.00 pEC₅₀, more preferably greater than 7.50 pEC₅₀, more preferably greater than 7.75 pEC₅₀, and even more preferably greater than 8.00 pEC₅₀.

[44] RARβ agonists include the fluorinated alkoxythiazoles previously described, such as:

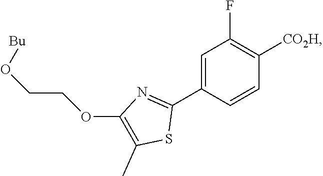

[45] 4′-Octyl-[1,1′-biphenyl]-4-carboxylic acid (65), Adapalene (67), BMS-231973, BMS-228987, BMS-276393, BMS-209641 (66), BMS-189453{4-[(1E)-2-(5,6-Dihydro-5,5-dimethyl-8-phenyl-2-naphthalenypethenyl]-benzoic acid} (68), CD2019 (6-[4-methoxy-3-(1-methylcyclohexyl)phenyl]naphthalene-2-carboxylic acid), compounds described in WO2008/064136 and WO2007009083, each of which is incorporated herein by reference in its entirety, and tazarotene (ethyl 6-[2-(4,4-dimethyl-3,4-dihydro-2H-1-benzothiopyran-6-yDethynyl] pyridine-3-carboxylate). Structures of some RARβ agonists are provided below:

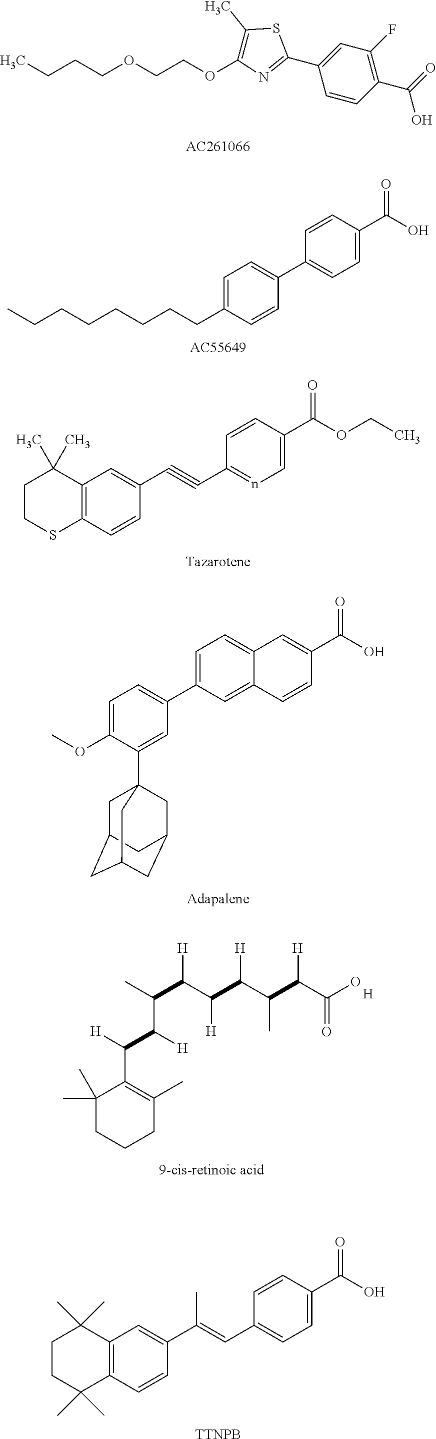

[52] RARβ agonists also include those disclosed in published PCT patent application WO2008/064136, WO2007/009083 and published U.S. patent application US2009/0176837, each of which is incorporated herein by reference in its entirety. The highly selective RARβ agonists AC261066 and AC55649, are highly isoform-selective agonists for the human RARβ₂ receptors as described in Lund et al. (2005, J. Med. Chem., 48, 7517-7519), which is incorporated herein by reference in its entirety. In a particular embodiment, RARβ₂ agonist is AC261066.

[53] RARβ₂ receptor agonists may also be selected from the following compounds or an ester thereof:

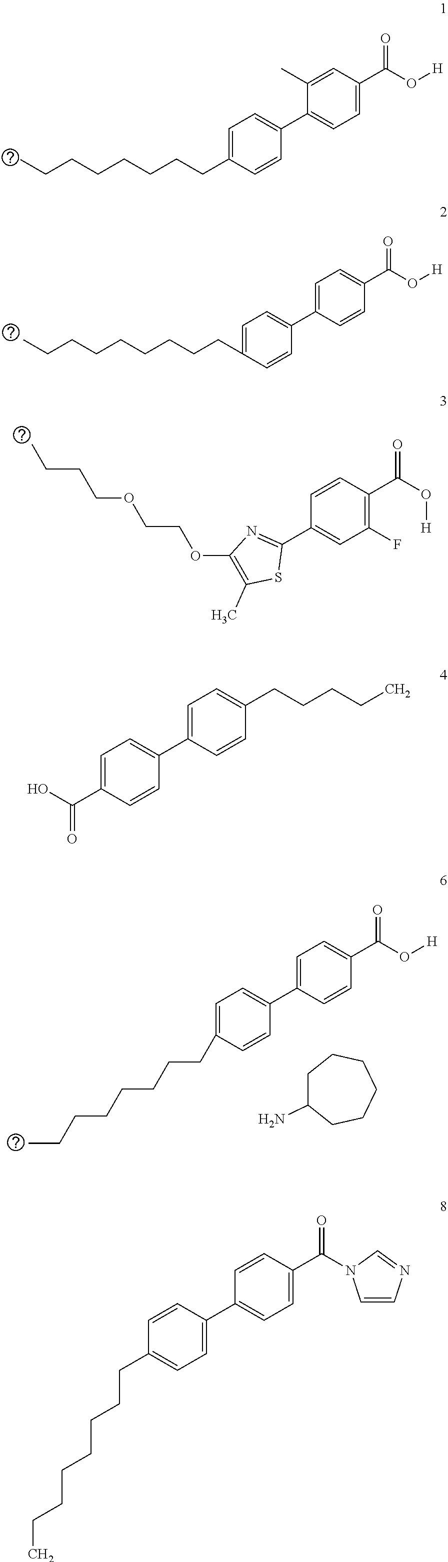

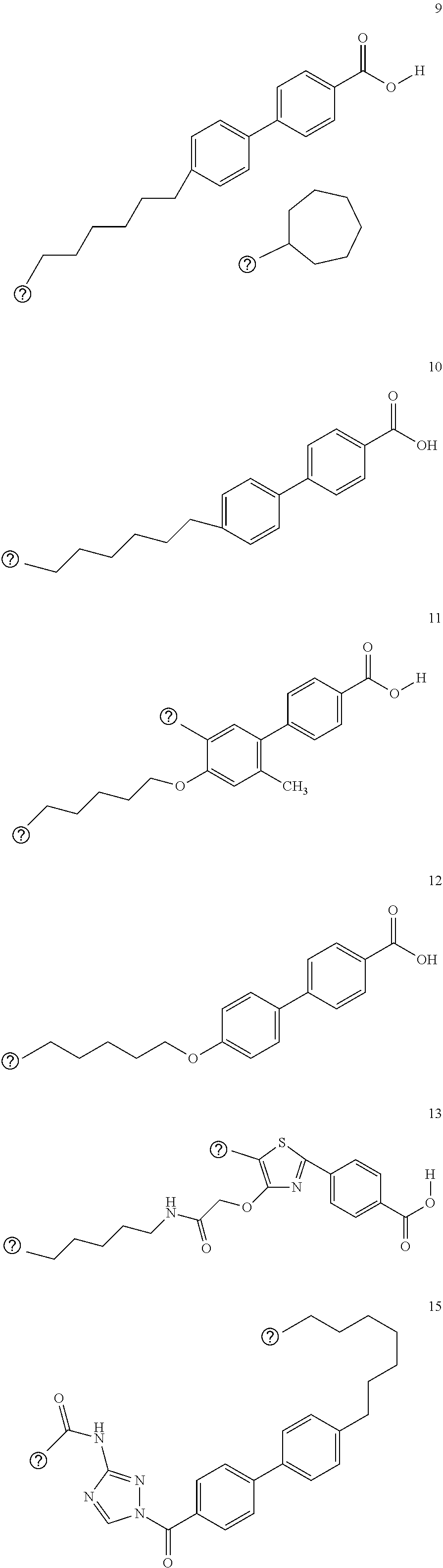

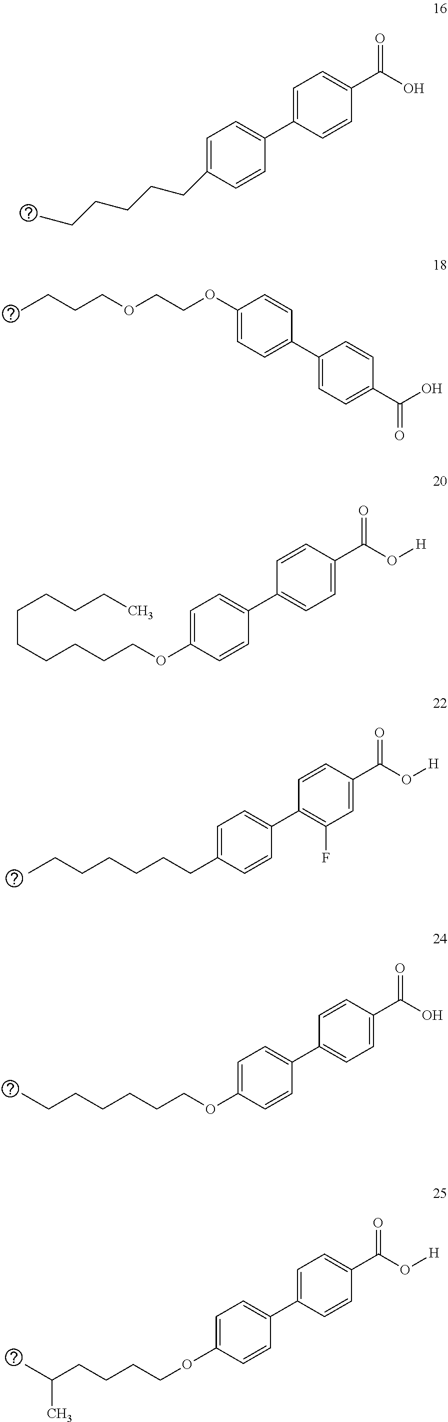 [SMILES: CCCCCCc1ccc(-c2ccc(C(=O)O)cc2)cc1 ; CCCCOCCOc1ccc(-c2ccc(C(=O)O)cc2)cc1 ; CCCCCCCCCCOc1ccc(-c2ccc(C(=O)O)cc2)cc1 ; CCCCCCCc1ccc(-c2ccc(C(=O)O)cc2F)cc1 ; CCCCCCCOc1ccc(-c2ccc(C(=O)O)cc2)cc1 ; CC(C)CCCCOc1ccc(-c2ccc(C(=O)O)cc2)cc1]

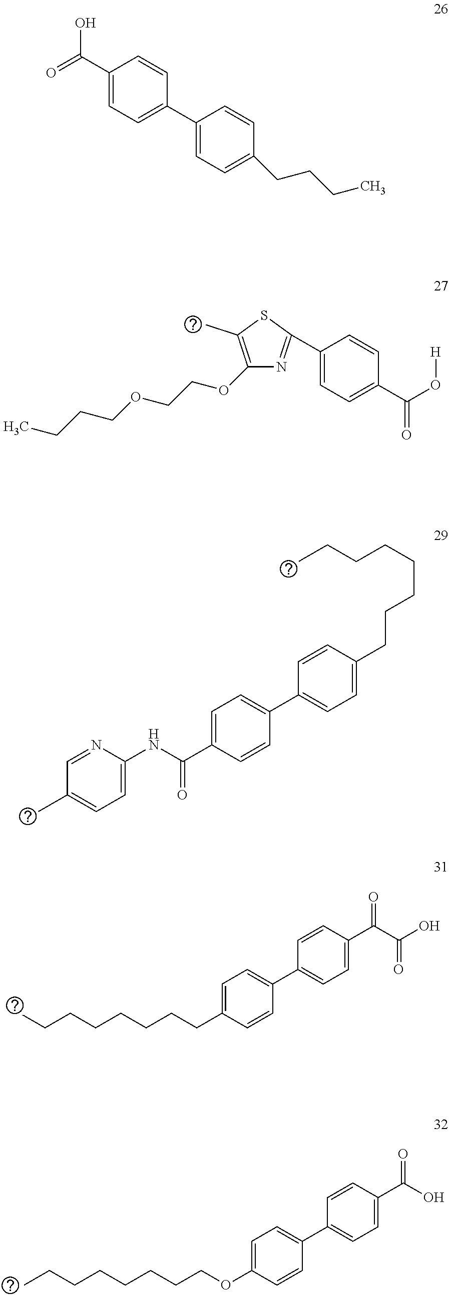

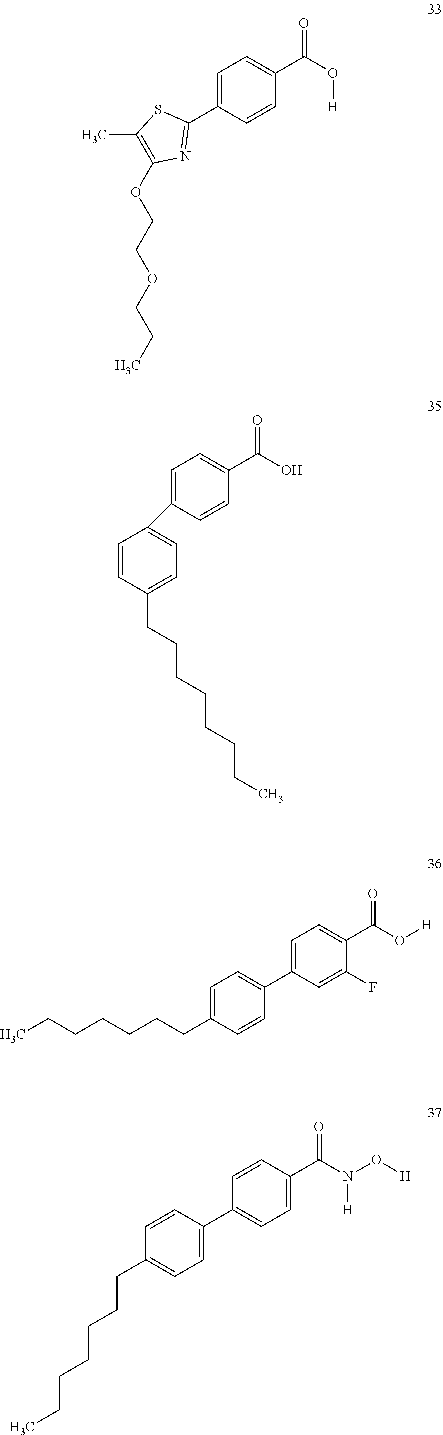 [SMILES: CCCOCCOc1nc(-c2ccc(C(=O)O)cc2)sc1C ; CCCCCCCCc1ccc(-c2ccc(C(=O)O)cc2)cc1 ; CCCCCCCc1ccc(-c2ccc(C(=O)O)c(F)c2)cc1 ; CCCCCCCc1ccc(-c2ccc(C(=O)NO)cc2)cc1]

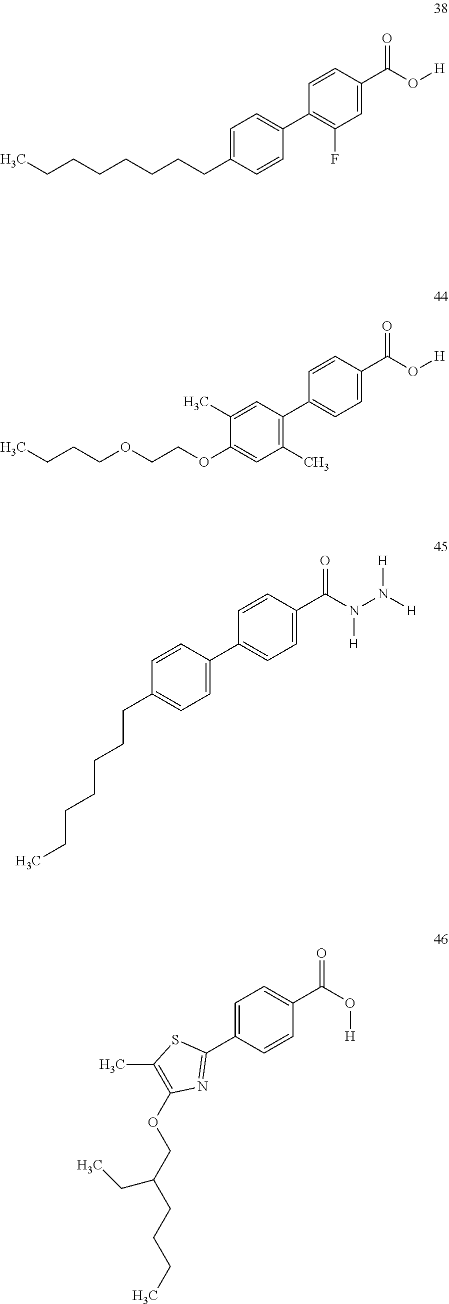 [SMILES: CCCCCCCCc1ccc(-c2ccc(C(=O)O)cc2F)cc1 ; CCCCOCCOc1cc(C)c(-c2ccc(C(=O)O)cc2)cc1C ; CCCCCCCc1ccc(-c2ccc(C(=O)NN)cc2)cc1 ; CCCCC(CC)COc1nc(-c2ccc(C(=O)O)cc2)sc1C]

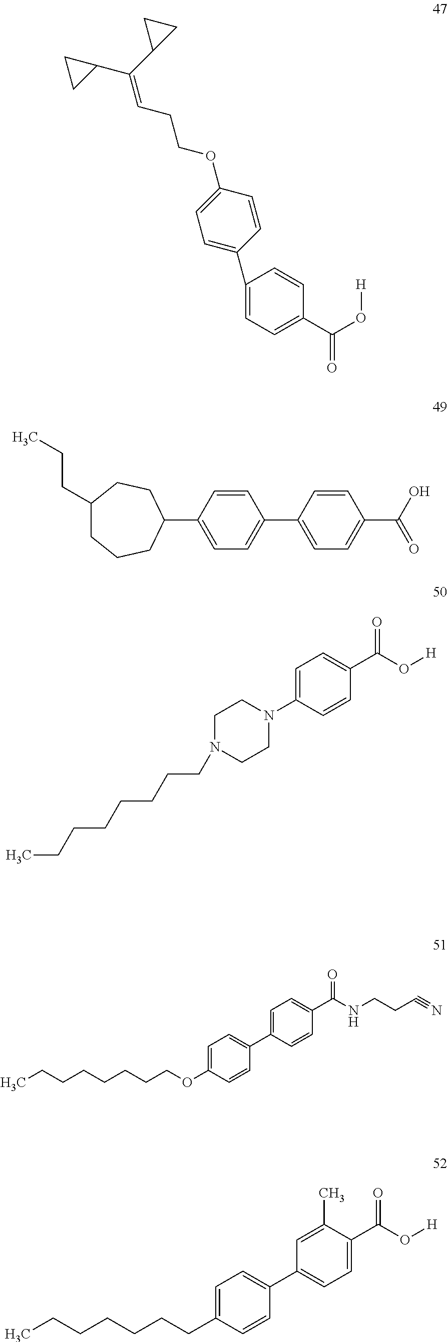 [SMILES: O=C(O)c1ccc(-c2ccc(OCCC=C(C3CC3)C3CC3)cc2)cc1 ; CCCC1CCCC(c2ccc(-c3ccc(C(=O)O)cc3)cc2)CC1 ; CCCCCCCCN1CCN(c2ccc(C(=O)O)cc2)CC1 ; CCCCCCCCOc1ccc(-c2ccc(C(=O)NCCC#N)cc2)cc1 ; CCCCCCCc1ccc(-c2ccc(C(=O)O)c(C)c2)cc1]

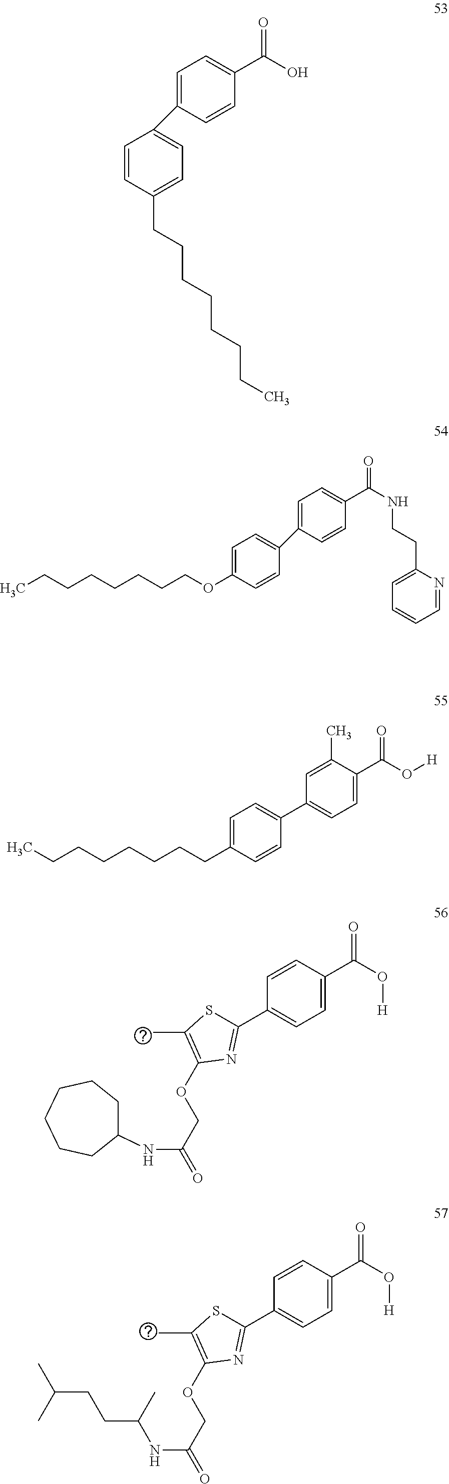 [SMILES: CCCCCCCCc1ccc(-c2ccc(C(=O)O)cc2)cc1 ; CCCCCCCCOc1ccc(-c2ccc(C(=O)NCCc3ccccn3)cc2)cc1 ; CCCCCCCCc1ccc(-c2ccc(C(=O)O)c(C)c2)cc1 ; Cc1sc(-c2ccc(C(=O)O)cc2)nc1OCC(=O)NC1CCCCCC1 ; Cc1sc(-c2ccc(C(=O)O)cc2)nc1OCC(=O)NC(C)CCC(C)C]

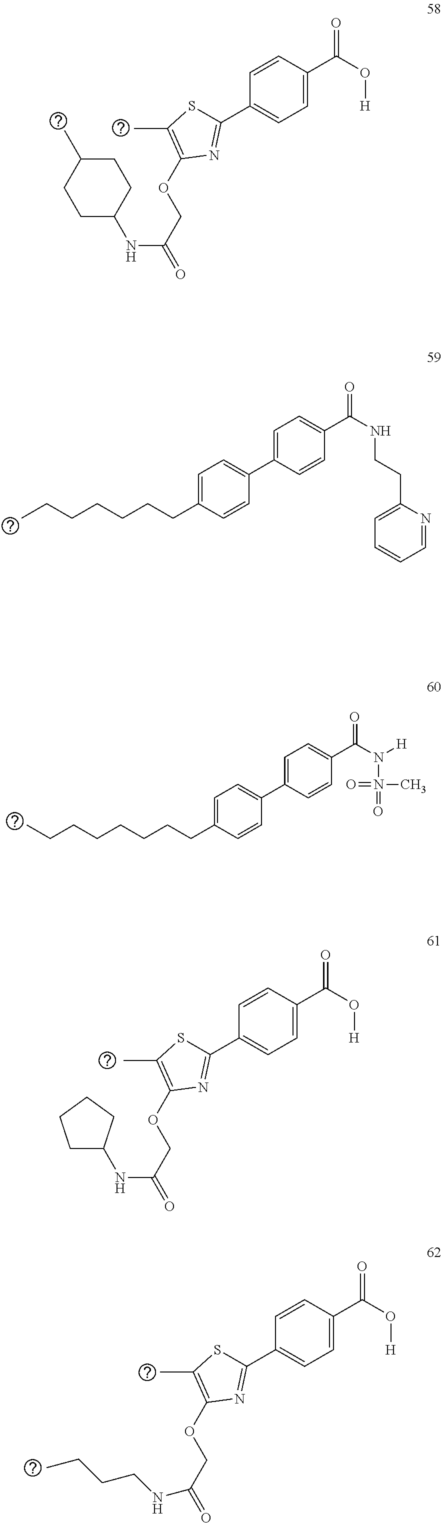

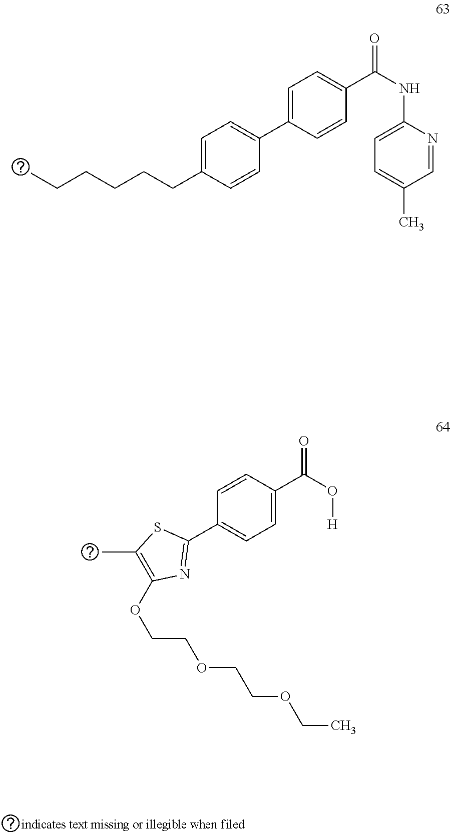 [SMILES: CCCCCCc1ccc(-c2ccc(C(=O)Nc3ccc(C)cn3)cc2)cc1 ; CCOCCOCCOc1nc(-c2ccc(C(=O)O)cc2)sc1C]

[54] The functional receptor assay, receptor selection and amplification may be performed as described in WO2007/009083, which is incorporated herein by reference in its entirety. For example, Technology (R-SAT) may be used to investigate the pharmacological properties of known and novel RARβ agonists useful for the present invention. R-SAT is disclosed, for example, in U.S. Pat. Nos. 5,707,798, 5,912,132, and 5,955,281, Piu et al., 2006, Beta Arrestin 2 modulates the activity of Nuclear Receptor RARβ₂ through activation of ERK2 kinase, Oncogen, 25(2):218-29 and Burstein et al., 2006, Integrative Functional Assays, Chemical Genomics and High Throughput Screening: Harnessing signal transduction pathways to a common HTS readout, Curr Pharm Des, 12(14): 1717-29, all of which are hereby incorporated herein by reference in their entireties, including any drawings.

[55] The relevant RARβ₂ receptor modulating activities of the above compounds are described in WO2007/009083, which is incorporated by reference herein in its entirety.

[56] The highly specific RARβ agonist, e.g., AC261066, can prevent hepatic steatosis and activation of HSCs, marked by decreased expression of α-SMA. AC261066 can significantly diminish hepatic gene expression of pro-inflammatory mediators tumor necrosis factor-alpha (TNFα) and monocyte chemotactic protein-1 (MCP-1).

[57] In one aspect, the invention provides a method for producing a cardioprotective effect on a heart of a subject in need thereof, comprising administering to the subject a therapeutically effective amount of a retinoic acid receptor-beta (RARβ) agonist.

[58] In various embodiments, the cardioprotective effect may be an attenuation of an infarct size in the heart. The cardioprotective effect may be a diminution of coronary norepinephrine spillover during reperfusion of the heart. The cardioprotective effect may be an alleviation of reperfusion arrhythmias. As one of ordinary skill in the art well knows, reperfusion arrhythmias may comprise ventricular tachycardia and/or ventricular fibrillation. The ventricular tachycardia and/or ventricular fibrillation may be present when the subject has a heart disease. In certain aspects, the ventricular tachycardia and/or ventricular fibrillation may be present prior to a myocardial infarction in the subject.

[59] In one aspect, the invention provides a method of attenuating or ameliorating cardiac contractile dysfunction in a subject having or having had a myocardial infarction MI or at risk of having a myocardial infarction, comprising administering to the subject a therapeutically effective amount of a selective retinoic acid receptor-β₂ (RARβ₂) agonist, e.g., AC261066 or AC55649, or a pharmaceutically acceptable salt, ester, amide, prodrug thereof, or a combination thereof. In some embodiments, attenuating or ameliorating cardiac contractile dysfunction includes increasing the post-MI (myocardial infarction) left ventricular ejection fraction. In some embodiments, attenuating or ameliorating cardiac contractile dysfunction includes improving post-MI (myocardial infarction) left ventricular fractional shortening.

[60] In another aspect, the invention provides a method of attenuating or ameliorating cardiac fibrosis in a subject having or having had a myocardial infarction (MI) or at risk of having a myocardial infarction, comprising administering to the subject a therapeutically effective amount of a selective retinoic acid receptor-β₂ (RARβ₂) agonist, e.g., AC261066 or AC55649, or a pharmaceutically acceptable salt, ester, amide, prodrug thereof, or a combination thereof.

[61] In an additional aspect, the invention provides a method of attenuating or ameliorating ventricular remodeling in a subject having or having had a myocardial infarction (MI) or at risk of having a myocardial infarction, comprising administering to the subject a therapeutically effective amount of a selective retinoic acid receptor-β₂ (RARβ₂) agonist, e.g., AC261066 or AC55649, or a pharmaceutically acceptable salt, ester, amide, prodrug thereof, or a combination thereof. In some embodiments, attenuating or ameliorating ventricular remodeling includes a decrease in end-diastolic left ventricular internal diameter.

[62] In some embodiments, the RARβ agonist may be administered before, during and/or after a myocardial infarction in the subject. In some embodiments, the RARβ agonist is administered chronically. In some embodiments, the RARβ agonist is administered acute The RARβ agonist may be administered chronically in a patient at risk for a myocardial infarction or for a shorter period for a patient at acute risk. Alternatively, the RARβ agonist may be administered during a myocardial infarction in the subject. In certain aspects, the RARβ agonist may be administered after a diagnosis of heart disease in the subject.

[63] In some aspects of the pharmaceutical formulations and methods provided herein, the RARβ agonist may be a synthetic selective retinoic acid β₂-receptor (RARβ₂) agonist or a pharmaceutically acceptable salt, ester, amide, prodrug thereof, or a combination thereof.

[64] In certain embodiments, the RARβ₂ agonist is AC261066 having a structure of Formula 1:

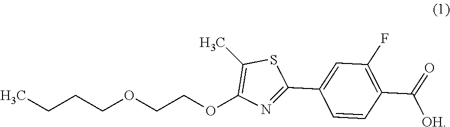 [SMILES: CCCCOCCOc1nc(-c2ccc(C(=O)O)c(F)c2)sc1C]

[65] In other embodiments, the RARβ₂ agonist is AC55649 having a structure of Formula 2:

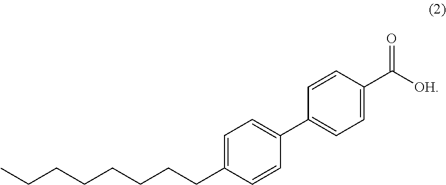 [SMILES: CCCCCCCCc1ccc(-c2ccc(C(=O)O)cc2)cc1]

[66] In alternate embodiments, the RARβ agonist is tazarotene having a structure of Formula 3:

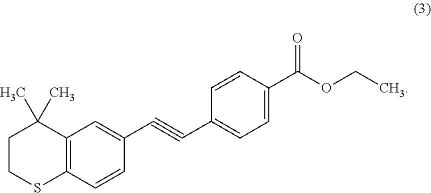 [SMILES: CCOC(=O)c1ccc(C#Cc2ccc3c(c2)C(C)(C)CCS3)cc1]

[67] In another aspect, the RARβ agonist is adapalene having a structure of Formula 4:

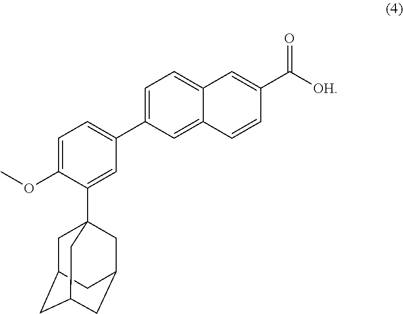 [SMILES: COc1ccc(-c2ccc3cc(C(=O)O)ccc3c2)cc1C12CC3CC(CC(C3)C1)C2]

[68] In still another aspect, the RARβ agonist is 4-[(E)-2-(5,6,7,8-Tetrahydro-5,5,8,8-tetramethyl-2-naphthalenyl)-1-propenyl]benzoic acid (TTNPB) having a structure of Formula 5:

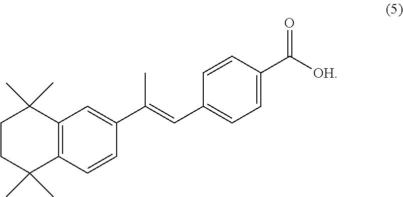 [SMILES: C/C(=C\c1ccc(C(=O)O)cc1)c1ccc2c(c1)C(C)(C)CCC2(C)C]

[69] In certain embodiments, the RARβ agonist may be administered three times daily. In other embodiments, the RARβ agonist may be administered in an amount from about 30 mg to about 200 mg per day.

[70] In other aspects, the RARβ agonist may be administered at a concentration of from about 0.1 mg to about 10 mg per 100 mL. The RARβ agonist also may be administered at a concentration from about 1 mg to about 3 mg per 100 mL.

[71] In various embodiments, the RARβ agonist may be administered orally. Alternatively, the RARβ agonist may be administered parenterally.

[72] Parenteral administration includes intravenous, subcutaneous, sublingual, buccal, nasal, intraarterial, intracardiac, intraarticular, transdermal, transmucosal, intramuscular, intraperitoneal, ophthalmic and/or topical administration.

[73] In other aspects, the presently provided methods further comprise administering to the subject a second therapeutic agent. The second therapeutic agent may be a second retinoic acid receptor-beta (RARβ) agonist. The second administered RARβ agonist may be an RARβ₂ agonist, for example, AC261066 having a structure of Formula 1:

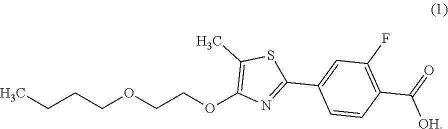 [SMILES: CCCCOCCOc1nc(-c2ccc(C(=O)O)c(F)c2)sc1C]

[74] In an additional embodiment, the second administered RARβ agonist may be an RARβ₂ agonist, for example, AC55649 having a structure of Formula 2:

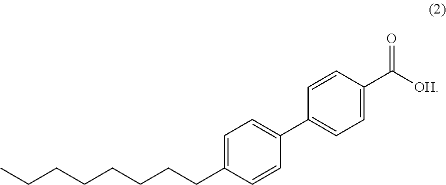 [SMILES: CCCCCCCCc1ccc(-c2ccc(C(=O)O)cc2)cc1]

[75] In another aspect, the invention provides a method for reducing myocardial injury to a heart of a subject in need thereof, comprising administering to the subject a therapeutically effective amount of a retinoic acid receptor-beta (RARβ) agonist. The myocardial injury may be an ischemia/reperfusion (UR) injury. The UR injury may be a myocardial infarction. In various aspects, administration of the RARβ agonist reduces the myocardial infarction size. In certain embodiments, the RARβ agonist is a synthetic selective retinoic acid β₂-receptor (RARβ₂) agonist or a pharmaceutically acceptable salt, ester, amide, prodrug thereof, or a combination thereof. The RARβ₂ agonist may have a structure of Formula (1), as shown above. Alternatively, the RARβ₂ agonist may have a structure of Formula (2), as shown above. In additional embodiments, the RARβ agonist may have a structure of any one of Formula (3), Formula (4), or Formula (5), each of which is shown above.

[76] In additional aspects of the method for reducing myocardial injury to a heart of a subject in need thereof, the method further comprises administering to the subject a second therapeutic agent. The second therapeutic agent may be a second retinoic acid receptor-beta (RARβ) agonist, and in particular, the second retinoic acid receptor-beta (RARβ) agonist may be an RARβ₂ agonist having a structure of Formula 1, as shown above, or an RARβ₂ agonist having a structure of Formula 2, as shown above.

[77] In another aspect, the invention provides a method for treating or alleviating chronic arrhythmia or reducing the chance of an arrhythmia occurring in a subject in need thereof, comprising administering to the subject a therapeutically effective amount of a retinoic acid receptor-beta (RARβ) agonist. The RARβ agonist may be a synthetic selective retinoic acid receptor β₂ (RARβ₂) agonist or a pharmaceutically acceptable salt, ester, amide, prodrug thereof, or a combination thereof. More specifically, the RARβ₂ agonist may have may be an RARβ₂ agonist having a structure of Formula 1, as shown above, or an RARβ₂ agonist having a structure of Formula 2, as shown above. In further embodiments, the RARβ agonist may have a structure of any one of Formula (3), Formula (4), or Formula (5), each of which is shown above. In certain aspects of the method for treating or alleviating chronic arrhythmia in a subject in need thereof, the method may further comprise administering to the subject a second therapeutic agent. The second therapeutic agent may be a second retinoic acid receptor-beta (RARβ) agonist, and in particular, the second retinoic acid receptor-beta (RARβ) agonist may be an RARβ₂ agonist having a structure of Formula 1, as shown above, or an RARβ₂ agonist having a structure of Formula 2, as shown above.

[78] In a further aspect, the invention provides a method for treating cardiac failure in a subject in need thereof, comprising administering to the subject a therapeutically effective amount of a retinoic acid receptor-beta (RARβ) agonist. The RARβ agonist may be a synthetic selective /specific retinoic acid 32-receptor (RARβ₂) agonist or a pharmaceutically acceptable salt, ester, amide, prodrug thereof, or a combination thereof. More specifically, the RARβ₂ agonist may have may be an RARβ₂ agonist having a structure of Formula 1, as shown above, or an RARβ₂ agonist having a structure of Formula 2, as shown above. In further embodiments, the RARβ agonist may have a structure of any one of Formula (3), Formula (4), or Formula (5), each of which is shown above. In certain aspects of the method for treating cardiac failure in a subject in need thereof, the method may further comprise administering to the subject a second therapeutic agent. The second therapeutic agent may be a second retinoic acid receptor-beta (RARβ) agonist, and in particular, the second retinoic acid receptor-beta (RARβ) agonist may be an RARβ₂ agonist having a structure of Formula 1, as shown above, or an RARβ₂ agonist having a structure of Formula 2, as shown above.

[79] In another aspect, the invention provides a pharmaceutical formulation comprising a therapeutically effective amount of a retinoic acid receptor-beta (RARβ) agonist and a pharmaceutically acceptable excipient. The RARβ agonist may be a synthetic selective retinoic acid β₂ receptor (RARβ₂) agonist or a pharmaceutically acceptable salt, ester, amide, prodrug thereof, or a combination thereof. In certain embodiments, the RARβ₂ agonist may have a structure of Formula 1, as shown above, or an RARβ₂ agonist having a structure of Formula 2, as shown above. In alternate aspects, the RARβ agonist may have a structure of any one of Formula (3), Formula (4), or Formula (5), each of which is shown above.

[80] The pharmaceutical formulation may comprise the RARβ agonist in an amount of from about 30 mg to about 200 mg. In certain aspects, the pharmaceutical formulation may comprise the RARβ agonist in an amount of from about 10 mg to about 100 mg. In various embodiments, the pharmaceutical formulation may comprise the RARβ agonist an amount of from about 10 mg to about 100 mg. Alternatively, the pharmaceutical formulation may comprise the RARβ agonist in an amount of from about 10 mg to about 67 mg. The pharmaceutical formulation may comprise the RARβ agonist in a concentration of from about 0.1 mg to about 10 mg per 100 mL. In other aspects, the pharmaceutical formulation may comprise the RARβ agonist in a concentration from about 1 mg to about 3 mg per 100 mL.

[81] The pharmaceutical formulation comprising the RARβ agonist, in particular, an RARP2 agonist, may be formulated with a pharmaceutically acceptable excipient for oral administration. In an alternate aspect, the pharmaceutical formulation may be formulated with a pharmaceutically acceptable excipient for parenteral administration. In other aspects, the pharmaceutical formulation may be formulated with a pharmaceutically acceptable excipient for intravenous, subcutaneous, sublingual, buccal, nasal, intraarterial, intracardiac, intraarticular, transdermal, transmucosal, intramuscular, intraperitoneal, ophthalmic or topical administration.

[82] In a further aspect, the invention provides a pharmaceutical formulation comprising a therapeutically effective amount of a retinoic acid receptor-beta (RARβ) agonist and a pharmaceutically acceptable excipient.

[83] As used herein, the term “pharmaceutically acceptable excipient” refers to those excipients which are, within the scope of sound medical judgment, suitable for use in contact with the tissues of humans and lower animals without undue toxicity, irritation, allergic response, and the like, and are commensurate with a reasonable benefit/risk ratio.

[84] The term “pharmaceutically acceptable excipient” includes any and all solvents, diluents, and other liquid vehicles, dispersion or suspension aids, surface active agents, isotonic agents, thickening or emulsifying agents, preservatives, solid binders, lubricants and the like, as suited to the particular dosage form desired. Remington: The Science and Practice of Pharmacy, 20th Ed., ed. A. Gennaro, Lippincott Williams & Wilkins, 2000, incorporated herein by reference in its entirety, specifically incorporated by reference in its entirety are disclosures of various excipients and carriers used in formulating pharmaceutically acceptable compositions and known techniques for the preparation thereof.

[85] The pharmaceutical compositions of the invention may be manufactured by methods well known in the art such as conventional granulating, mixing, dissolving, encapsulating, lyophilizing, or emulsifying processes, among others. Compositions may be produced in various forms, including granules, precipitates, or particulates, powders, including freeze dried, rotary dried or spray dried powders, amorphous powders, tablets, capsules, syrup, suppositories, injections, emulsions, elixirs, suspensions, or solutions. Formulations may optionally contain excipients, including but not limited to, solvents, diluents, and other liquid vehicles, dispersion or suspension aids, surface active agents, pH modifiers, isotonic agents, thickening or emulsifying agents, stabilizers and preservatives, solid binders, lubricants, and the like, as suited to the particular dosage form desired.

[86] The RAR agonist, and in particular a RARβ₂ agonist, may be administered by any method known to one skilled in the art. For example, an RAR agonist, and in particular a RARβ₂ agonist, may be administered orally or parenterally.

[87] The term “parenteral” as used herein includes subcutaneous, intravenous, intramuscular, intra-articular, intra-synovial, intrasternal, intrathecal, intrahepatic, intralesional and intracranial injection or infusion techniques. The pharmaceutical compositions and formulations may be administered orally, intravenously, or subcutaneously. The formulations of the invention may be designed to be short-acting, fast-releasing, or long-acting. Still further, compounds may be administered locally, e.g., intracardially, rather than by systemic means, such as administration (e.g., by injection) to a heart of a subject having ischemia or a myocardial infarction.

[88] Liquid dosage forms for oral administration include, but are not limited to, pharmaceutically acceptable emulsions, microemulsions, solutions, suspensions, syrups, and elixirs. In addition to the active compounds, the liquid dosage forms may contain inert diluents commonly used in the art such as, for example, water or other solvents, solubilizing agents and emulsifiers. Besides inert diluents, the oral compositions can also include adjuvants such as wetting agents, emulsifying and suspending agents, sweetening, flavoring, and perfuming agents.

[89] Injectable pharmaceutical formulations comprising an RARβ agonist, specifically an RARβ₂ agonist, for example, sterile injectable aqueous or oleaginous suspensions may be formulated according to the known art using suitable dispersing or wetting agents and suspending agents. The sterile injectable pharmaceutical formulation may also be a sterile injectable solution, suspension or emulsion in a nontoxic parenterally acceptable diluent or solvent, for example, as a solution in 1,3-butanediol. Among the pharmaceutically acceptable vehicles and solvents that may be employed are water, Ringer's solution, U.S.P. and isotonic sodium chloride solution. In addition, sterile, fixed oils are conventionally employed as a solvent or suspending medium. For this purpose, any bland fixed oil may be employed, including but not limited to, synthetic mono- or diglycerides. In addition, fatty acids such as oleic acid are used in the preparation of injectables.

[90] Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar, calcium carbonate, potato or tapioca starch, alginic acid, certain silicates, and sodium carbonate, e) solution retarding agents such as paraffin, 0 absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents such as phosphates or carbonates.

[91] Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art.

[92] The RAR agonist, in particular an RARβ₂ agonist, of the present invention may be used in combination with a second drug or therapeutic agent, wherein the second drug or second therapeutic agent is administered in a therapeutically effective amount to a subject in need thereof. The second therapeutic agent may be a second retinoic acid receptor-beta (RARβ) agonist. The second drug or therapeutic agent include, but not be limited to, an antiplatelet medication to prevent formation of blood clots in the arteries (such as aspirin, thienopyridines (e.g., clopidogrel or prasugrel is used instead of aspirin in patients who have an aspirin allergy), and the glycoprotein IIb/IIIa inhibitors), an anticoagulant medication to prevent growth of blood clots in the arteries (such as intravenous or subcutaneous heparin, subcutaneous low molecular weight heparin, and oral warfarin (Coumadin) or direct thrombin inhibitors (e.g., rivaroxaban (Xarelto) and dabigatran (Pradaxa)), clot-dissolving medications to open blocked arteries (such as fibrinolytic or thrombolytic medications, (e.g., intravenous administration of tissue plasminogen activator (TPA) or TNK, which can open up to 80% of acutely blocked coronary arteries)), medications to decrease the need for oxygen by the heart's muscle (such as nitroglycerine, which dilates coronary arteries), medications to prevent abnormal heart rhythms (such as antiarrhythmic drugs (e.g., amiodarone, and lidocaine) or beta blockers for tachycardia (e.g., acebutol and propranolol)) or a combination thereof when medically advisable in the professional judgement of one ordinarily skilled in the art of treating and/or preventing ischemia and myocardial infarctions.

[93] Additional treatment may include coronary angiography with either percutaneous transluminal coronary angioplasty (PTCA) with or without stenting to open blocked coronary arteries. Supplemental oxygen may be administered to the subject to increase the supply of oxygen to the heart's muscle.

[94] All patents and literature references cited in the present specification are hereby incorporated by reference in their entirety.

### EXAMPLES

### Example 1

### Methods

I/R in Ex-vivo Mouse Hearts

[95] Wild type (WT) C57/BL6 and ApoE^{-/-}male mice (C57/13L6 background, from Jackson Labs, Bar Harbor, ME) were maintained on a regular laboratory chow diet (Con diet, # 5053, Pico Diet, PicoLab Rodent Diet, LabDiet, St. Louis, Mo.). Six weeks after birth, another group of ApoE^{-/-} mice received, in addition to their chow diet, drinking water containing 3.0 mg AC261066/100 mL in 0.1% DMSO/H₂O for 6 weeks. Twenty minutes after a heparin injection (100 I.U., i.p.) to avoid blood clotting, mice were anesthetized with CO₂ vapor and humanely killed by cervical dislocation while under anesthesia (Institutional Animal Care and Use Committee approved). Hearts were quickly excised and cooled in ice-cold Krebs-Henseleit (KH) solution (composition, mM: NaCl 120; KCl4.71; CaCl₂ 2.5; MgSO₄ 1.2; KH₂PO₄ 1.2; NaHCO₃ 84.01; Dextrose 11.1; Pyruvic acid 2.0; EDTA 0.5), equilibrated with 95% O₂+5% CO₂. Hearts were then cannulated via the aorta with a 20-gauge needle and perfused in a Langendorff apparatus (Radnoti, Monrovia, Calif.) at constant pressure (100 cm H₂O) with KH solution at 37° C. Two needle electrodes were attached to the surface of the right atrium and left ventricular apex for ECG recordings. ECG was recorded online (sample frequency of 1 kHz) and arrhythmia duration was analyzed using Powerlab/8SP (ADInstruments, Colorado Springs, Colo.). Following a 20-minute stabilization, all hearts were subjected to 40-minute normothermic global ischemia, induced by complete cessation of coronary perfusion, followed by 120-minute reoxygenation (reperfusion) with KH solution (I/R). I/R-injured areas (i.e., infarct size) were analyzed at the end of reperfusion. Coronary flow was measured by timed collections of the effluent every 2 minutes, and samples were assayed for norepinephrine (NE) by high-performance liquid chromatography with electrochemical detection as previously described.

[96] Experiments were also performed on WT C57/BL6 mice, maintained on either a standard laboratory chow (Control diet) (# 5053, Pico Diet) or a high-fat diet (I-IFD) with 45% kcal from fat, (#58125, Test Diet) for 3-5 months). Six weeks after the start of the HFD, these mice received either drinking water containing 0.1% DMSO (vehicle) or water/0.1% DMSO containing AC261066 (3.0 mg/100 mL). As powder, AC261066 is stable at room temperature, according to the manufacturer. The HPLC results showed that AC261066 is stable in the drinking water for at least one week at room temperature. The half-life in the heart is not known. The scientist performing the ex-vivo I/R procedure was blinded to the identities of the groups of mice.

### TTC Staining for Ischemic Area in Mouse Hearts

[97] At the end of 120-minute reperfusion, hearts were cut (6-7 sections/heart, 2-mm thick), incubated in an aqueous solution of 2,3,5-triphenyltetrazolium chloride (1% w/v) (TTC, Sigma-Aldrich, St. Louis, Mo.) for 20 minutes at 37° C., and transferred to a formaldehyde solution (10% v/v) for overnight fixation. Heart slices were photographed with a 16×magnification and analyzed by computerized morphometry using Image J software (NIH) to measure infarct sizes (expressed as percentage of ischemic vs. total left ventricular area). Infarct size was measured in every slice and then averaged for each single heart. The most representative sections were chosen for illustration.

### Malondialdehyde (MDA) Staining

[98] Cryo-sectioning. At the end of I/R, mouse hearts were fixed in 4% paraformaldehyde in PBS overnight at 4° C., and then incubated in 30% sucrose in PBS overnight at 4° C. Tissues were embedded in optimal cutting temperature (OCT) compound and subjected to cryo-sectioning. The sections were 15-gm thick and stored at −80° C. prior to immunostaining.

[99] Immunostaining. Frozen sections were dried at room temperature before staining. Briefly, the sections were washed in PBS, blocked in 10% goat serum plus 0.02% Triton X-100 in PBS for 30 minutes at room temperature, followed by incubation with an anti-malondialdehyde (MDA) antibody (1:200, Abcam, cat# 6463, lot # GR3191333-3, Cambridge, MA) overnight at 4° C. Sections were then incubated with a goat anti-rabbit IgG secondary antibody at room temperature for 1 hour (cat # B40962, ready to use, Thermo Fisher Scientific, Eugene, Oreg.). As a negative control, sections were stained without incubation with the primary antibody. Signals were visualized based on a peroxidase-detection mechanism with 3,3-diaminobenzidine (DAB) (Product# 34002, Thermo Fisher Scientific, Rockford, Ill.) used as the substrate. Six to eight representative areas of each heart section from 3-4 mice per group were photographed and analyzed.

### Toluidine Blue Staining of Mast Cells (MC)

At the end of 120-minute reperfusion, hearts were cut and processed for frozen sections. Heart sections (15-gm thick) were stained with toluidine blue (0.5%) to visualize MC under transmitted light. MC were identified with a 60X magnification. Intact and degranulated MC were counted in the analyzed sections, and MC degranulation was calculated as a percentage of degranulated MC over total MC. Three sections of each heart from 3-4 mice per group were analyzed.

### Lipid Panel Measurements

Lipid panel measurements were carried out using the CardioChek® PA analyzer (PTS Diagnostics, Indianapolis, Ind.). Briefly, 40 gl of mouse tail blood were applied to test strips to measure the levels of total cholesterol, high-density lipoprotein (HDL) cholesterol, low-density lipoprotein (LDL) cholesterol, and triglycerides. Statistical differences among the groups were calculated using one-way analysis of variance (ANOVA), followed by the Bonferroni Correction for post hoc analysis.

### Statistical Analysis of Data

Statistical analysis was performed with GraphPad Prism 7.0 software (San Diego, Calif., USA). All data are reported as means ±SEM. Statistical analysis was performed only when a minimum of n=5 independent samples was acquired, and was performed with unpaired t-test when comparing two different groups and with one-way ANOVA followed by Dunnett's post-hoc test when comparing more than two groups of data. Data and statistical analysis comply with the recommendations on experimental design and analysis in Pharmacology. Data were considered statistically significant when a value of at least p<0.05 was achieved.

### Example 2

### The RARβ₂ Agonist AC261066 Decreases NE Overflows, Reperfusion Arrhythmias and Infarct Sizes in Murine ApoE^{-/-}Hearts Subjected to Ex-Vivo UR

Since high blood cholesterol levels are a major factor in myocardial ischemia, first investigated was whether the RARβ₂ agonist AC261066 influences relevant parameters of I/R-induced cardiac dysfunction, such as NE release, arrhythmia severity, and infarct size in hypercholesterolemic ApoE^{-/-}mice. For this, spontaneously beating Langendorff-perfused mouse hearts were subjected to 40-minute global ischemia followed by 120-minute reperfusion (I/R). In control WT hearts, it was found that NE overflows during reperfusion amounted to ˜80 μmol/g, ventricular arrhythmias (tachycardia and fibrillation, VT/VF) lasted ˜50 seconds, and infarct sizes comprised ˜25% of the left ventricle. In ApoE^{-/-}hearts subjected to I/R, NE overflows, VT/VF durations and infarct sizes were each ˜60% greater than in WT hearts. Notably, in hearts from ApoE^{-/-}mice treated orally for 6 weeks with AC261066 and then subjected to I/R ex vivo in the absence of AC261066, NE overflows, VT/VF durations, and infarct sizes were markedly reduced by ˜35-45% as compared to those in untreated ApoE^{-/-}hearts subjected to I/R (FIG. 1). Notably, coronary flow in ApoE^{-/-}hearts (3.025 ±0.46 mL/min) did not differ from that of ApoE^{-/-}hearts treated with AC261066 (2.092 ±0.25 mL/min). These findings suggest that signaling via RARβ₂ exerts protective effects in a genetic hypercholesterolemic ex-vivo I/R heart model.

### Example 3

### The RARβ₂ Agonist AC261066 Limits the Increase in Oxidative Stress in Murine ApoE^{-/-} Hearts Subjected to Ex-Vivo UR

Since the hearts from mice treated orally with AC261066 displayed protective features in this ex-vivo hypercholesterolemic I/R model in the absence of AC261066 in vitro, the inventors questioned whether these cardioprotective effects resulted from an attenuation of I/R-induced oxidative stress. Accordingly, whether the RARβ₂ agonist AC261066 influences the production of malondialdehyde (MDA), a classical marker of oxidative stress, was investigated. It was found that a 6-week oral treatment with AC261066 markedly reduced the level of MDA in hearts from ApoE^{-/-}mice subjected to UR as compared to their untreated controls; this reduction amounted to ˜60% (FIG. 2). These findings suggested that the protective effects provided by the drug AC261066 in ApoE^{-/-}hearts likely result at least in part from a reduction in I/R-induced oxidative stress.

### Example 4

### The RARβ₂ Agonist AC261066 Reduces Mast Cell (MC) Degranulation in Murine ApoE^{-/-}Hearts Subjected to Ex-Vivo UR

I/R-induced cardiac dysfunction is known to be associated with local MC degranulation and consequent release of noxious mediators, elicited by toxic aldehydes produced in the setting of oxidative stress (Koda et al., 2010). Thus, whether the cardioprotection afforded by AC261066 in I/R is accompanied by a reduction in MC degranulation we determined. Frozen sections of WT, ApoE^{-/-}and AC261066-treated ApoE^{-/-}murine hearts subjected to I/R were stained with toluidine blue to identify MC and assess their degranulation. We found that in hearts from WT mice, I/R elicited MC degranulation which amounted to ˜30% of the total MC number. In hearts from ApoE^{-/-} mice, I/R-induced MC degranulation increased to ˜50%, but in AC261066-treated ApoE^{-/-}hearts I/R-induced MC degranulation was reduced by ˜30% as compared to that in untreated ApoE^{-/-}hearts (FIG. 3). These data indicate that the protective effects provided by AC261066 in ApoE^{-/-}hearts subjected to ex-vivo UR may result from a decrease in MC degranulation elicited by products of oxidative stress.

### Example 5

### The RARβ₂ agonist AC261066 Decreases NE Overflows, Reperfusion Arrhythmias, and Infarct Sizes in HFD-Fed Murine Hearts Subjected to Ex-Vivo UR

The present studies with ApoE^{-/-}mice indicated that AC261066 exerts cardioprotective anti-UR effects. Therefore, we next investigated whether cardioprotection also occurs in hearts from WT C57BL6 mice fed a high-fat diet (HFD), which is known to enhance oxidative stress and cause cardiac dysfunction (Zeng et al., 2015). For this testing, spontaneously beating, Langendorff-perfused hearts from chow, HFD- and HFD +AC261066-fed mice were subjected to 40-minute global ischemia followed by 120-minute reperfusion (YR). In hearts from chow-fed mice, NE overflows during reperfusion amounted to ˜60 μmol/g, ventricular arrhythmias (tachycardia and fibrillation, VT/VF) lasted ˜60 seconds, and infarct sizes comprised ˜25% of left ventricles (FIG. 4). In hearts from HFD-fed mice subjected to I/R, NE overflows were as large as in hearts from chow-fed WT, and VT/VF durations and infarct sizes were ˜4- and 2-fold greater than in chow-fed WT hearts. In hearts from HFD-fed mice treated orally with AC261066 prior to ex-vivo I/R, NE overflows, VT/VF durations, and infarct sizes were markedly reduced by ˜45-65% as compared with those in untreated HFD hearts (FIG. 4). Notably, coronary flow in HFD hearts (3.02 ±0.19 mL/min) did not differ from that of HFD hearts treated with AC261066 (3.015 ±0.27 mL/min). Thus, the RARβ₂ agonist AC261066 also displays protective effects in a non-genetic, obese mouse ex-vivo I/R heart model.

### Example 6

### Oral AC261066 Treatment does not Reduce Total Cholesterol, Triglyceride, HDL and LDL Blood Levels in ApoE^{-/-}Mice

There were no significant differences in body weight among WT, ApoE^{-/-}and ApoE^{-/-}mice treated orally with AC261066 (FIG. 5, panel A). Total cholesterol, triglycerides, HDL, and LDL blood levels were markedly increased in ApoE^{-/-}mice as compared with WT controls, whereas HDL levels were slightly reduced in ApoE^{-/-}and AC261066-treated ApoE^{-/-}mice when compared to WT. Most importantly, oral treatment of ApoE^{-/-}mice with AC261066 did not modify total cholesterol, triglycerides, HDL and LDL blood levels (FIG. 5, panel B), indicating that the blood lipid profile does not play a major role in the cardioprotective effects of this RARβ₂ selective agonist.

The purpose of the present examples was to characterize the effects of a RARβ₂-selective, synthetic agonist, AC261066, in an ex-vivo I/R injury model in the heart. The inventors chose to test the effects of AC261066 in two dysmetabolic murine models because hyperlipidemic states are known to be causally associated with myocardial ischemia and oxidative stress, and also because it was previously discovered that AC261066 decreases oxidative stress in the liver, pancreas and kidneys of HFD-fed mice. The inventors found that a 6-week oral treatment with AC261066 in both genetically hypercholesterolemic (ApoE^{-/-}) and obese (HFD-fed) mice exerts protective effects when their hearts are subsequently subjected to I/R ex vivo in the absence of added drug. Most importantly, this cardioprotection ensued without any major changes in the hyperlipidemic state, indicating that the cardioprotective effects of this RARβ₂ selective agonist do not derive from hypothetical modification of the blood lipid profile (see FIG. 5). Furthermore, although RAR and RXR activation has been shown to relax resistance vessels via the endothelium-dependent NO-cGMP pathway, AC261066-induced cardioprotection was not associated with an increase in coronary flow, since treatment with AC261066 did not modify coronary flow in hearts from ApoE^{-/-}and HFD-fed mice.

Langendorff-perfused mouse hearts subjected to UR undergo a sizeable infarct of the left ventricle. A prominent characteristic of AC261066-afforded protection in our ex-vivo heart model was the reduction in I/R-induced infarct size in the hearts of both ApoE^{-/-}and HFD-fed WT mice. In as much as the extent of myocardial injury associated with UR results at least in part from oxidative stress and formation of toxic aldehydes, the inventors measured MDA levels, a known maker of oxidative stress, in post-I/R hearts, and found them to be significantly reduced in sections from AC261066-treated ApoE^{-/-}as compared with untreated ApoE^{-/-}control mice. Accordingly, this RARf32-selective agonist most likely reduces I/R-induced oxidative stress and thus, infarct size. Since the production of oxygen radicals and toxic aldehydes occurs primarily at the cardiomyocyte mitochondrial level, it is believed that intranuclear RARβ₂ activation recruits a yet-to-be uncovered signaling pathway which ultimately results in oxidative stress reduction. The AC261066-induced reduction in infarct size results from a decrease in apoptosis, favored by the reduction in oxidative stress, or involves other protective mechanisms.

Cardiac UR is accompanied by systemic and local sympathetic neural activation, which characteristically results in abundant NE release in the heart. I/R-induced activation of a local renin-angiotensin system (RAS) is a major contributor to this enhancement of NE release. The inventors found that NE coronary spillover was significantly increased during reperfusion in chow- and HFD-fed hearts, as well as in WT and ApoE^{-/-}hearts. A novel finding of the inventors was that oral treatment with AC261066 markedly curtailed I/R-induced NE overflow in the hearts from both ApoE^{-/-}and HFD-fed mice. Although it has not been previously demonstrated, it is plausible that RARβ₂ activation directly interferes with NE exocytosis from sympathetic nerve endings. Yet, a major mechanism of this protective effect probably derives from a diminished activation of local cardiac RAS, likely a result of the decreased MC degranulation also elicited by AC261066 treatment. Indeed, enzymatically active renin is present in cardiac MC, and renin release during UR constitutes the first step of local RAS activation that culminates in angiotensin-induced cardiac dysfunction, including excessive NE release.

While not intending to be bound by any particular mechanism of operation, it is believed that the reduction in NE release which occurred in the hearts of AC261066-treated mice also contributed to the decrease in infarct size. Indeed, I/R-induced cardiac injury is known to be enhanced by the vasoconstriction and increased oxygen demand associated with hyperadrenergic states. Concomitantly, RAS activation and increased angiotensin production likely contributed to the enhancement of cardiac injury, given the recognized capacity of angiotensin to promote the formation of oxygen radicals.

Whether AC261066 treatment decreased the I/R-induced degranulation of cardiac MC by one or more mechanisms directly involving MC exocytotic pathways remains to be determined. Nonetheless, given the well-known capability of oxygen radicals and toxic aldehydes to degranulate MC, while not intending to be bound by any particular mechanism of operation, it is believed that the AC261066-induced reduction in MC degranulation results from the attenuation of oxidative stress and toxic aldehyde formation by this selective RARβ₂ agonist.

Preeminent in the cardioprotective effects of AC261066 in our ex-vivo murine UR model was an alleviation of reperfusion arrhythmias, characterized by an abbreviation of ventricular tachycardia and fibrillation. Given the notorious arrhythmogenic effects of catecholamines, it is likely that the attenuation of NE release from UR hearts of the AC261066-treated mice played a major role in the anti-arrhythmic effects of AC261066. At the same time, since oxygen radicals are known to elicit cardiac arrhythmias by multiple mechanisms, it is likely that the AC261066-induced reduction in oxidative stress contributed to the alleviation of reperfusion arrhythmias. In addition, since angiotensin is highly arrhythmogenic, both directly and via oxygen radical production, the AC261066-induced reduction of MC degranulation, and thus of renin release and RAS activation, is likely to have contributed to its anti-arrhythmic effects.

In conclusion, treatment of mice with the selective RARβ₂ agonist AC261066 afforded cardioprotection in their ex-vivo hearts subjected to I/R. Cardioprotection consisted of an attenuation of infarct size, diminution of NE spillover, and alleviation of reperfusion arrhythmias. This cardioprotection was associated with a reduction in oxidative stress and MC degranulation.

While not intending to be bound by any particular mechanism of operation, it is believed that the reduction in myocardial injury and adrenergic activation, as well as the antiarrhythmic effects, result at least in part from decreased formation of oxygen radicals and toxic aldehydes known to elicit the release of MC-derived renin, promoting the activation of local RAS leading to enhanced NE release and reperfusion arrhythmias. In as much as these beneficial effects of AC261066 occurred at the ex-vivo level following oral drug treatment, the present data show that AC261066 is not only as a drug to reduce I/R injury of the heart, but also a drug for patients affected by other cardiovascular ailments, such as chronic arrhythmias and cardiac failure.

### EXAMPLE 7

### A Retinoic Acid Receptor P2 Agonist Improves Cardiac Function in a Heart Failure Model

Myocardial fibrosis is a key feature of heart failure (HF), contributing significantly to contractile dysfunction. An increased production of reactive oxygen species (ROS) is a recognized critical cause of myocardial fibrosis. Vitamin A (retinol), retinoic acid, and related metabolites, collectively known as retinoids, exert several effects on heart development and play a major role in cardiac regeneration in zebrafish models. A selective retinoic acid receptor (RAR) β₂ agonist, AC261066, was shown to be cardioprotective, leading to a diminution of norepinephrine spillover and significantly alleviating reperfusion arrhythmias in obese (i.e., high fat diet—fed) and genetically hypercholesterolemic (i.e., Apolipoprotein E (ApoE) knockout) mice. These cardioprotective changes were associated with a reduction in oxidative stress.

Without intending to be bound to theory, the inventors hypothesized that AC261066 can attenuate the development of myocardial contractile failure in HF by decreasing oxidative stress and consequent fibrotic response. This hypothesis was tested using an established murine model of ischemic HF, obtained by permanent occlusion of the left anterior descending (LAD) coronary artery, in which cardiac function is influenced by left ventricular maladaptive remodeling. Permanent LAD occlusion typically results in HF 3-4 weeks postsurgery and is widely used as a model of ventricular remodeling after myocardial infarction (MI), especially because it reproduces human HF with reduced ejection fraction.

This Example shows that oral treatment of mice with the RARβ₂ agonist AC261066, after induction of permanent LAD coronary artery occlusion, attenuated the decline in left ventricular ejection fraction (LVEF), decreased the development of oxidative stress in cardiomyocytes, and reduced interstitial fibrosis. Based on further experiments conducted in cardiac murine fibroblasts subjected to hypoxia, the cardioprotective effects of AC261066 are proposed to originate from actions on both cardiomyocytes and fibroblasts.

### Materials and Methods

In Vivo Studies

MI was obtained via ligation of the LAD coronary artery, performed as previously reported (Santulli et al., 2015; Kushnir et al., 2018). Briefly, male mice (3 months old) were anesthetized with isoflurane (4% for induction, 2% for maintenance), the heart was exposed via a left thoracotomy, and a permanent knot was tied around the LAD coronary artery. After surgery, mice were randomized to receive AC261066 (3 mg/100 mL) in drinking water or vehicle.

To assess cardiac function and remodeling after surgery, animals underwent a transthoracic ultrasound procedure, as previously reported (Santulli et al., 2012; Santulli et al., 2015). Briefly, echocardiography was performed using a small-animal high-resolution imaging system (Vevo2100, FUJIFILM VisualSonics, Toronto, Canada) at the Albert Einstein College of Medicine. The mice were anesthetized by isoflurane inhalation (induction: 4%) and maintained by mask ventilation (isoflurane 1.5%). Fur was removed from the chest by application of a depilatory cream (Veet, Reckitt Benckiser, Slough, UK) to gain a clear image. The mice were placed in a shallow left lateral decubitus position on a heated platform. All measurements were averaged on at least 10 consecutive cardiac cycles per experiment. Vevo LAB software (FUJIFILM VisualSonics) was used to acquire images and to assess cardiac morphology and function. Animals were maintained in a specific pathogen-free facility at Albert Einstein College of Medicine in New York City, N.Y., and the protocol for surgery and echocardiography in rodents was approved by the Einstein Animal Care Committee in accordance with the Association for Assessment and Accreditation of Laboratory Animal Care international guidelines. All animals were of the C57/BL6J background strain. All experiments were performed in a blinded fashion. Serum concentration of troponin I was measured 24 hours postsurgery using a commercially available immunoassay kit (Lifespan Bioscience, Seattle, WA) to confirm that the infarct size achieved by surgery was similar among groups.

### Ex Vivo and In Vitro Experiments

The level of malondialdehyde (MDA) in the left ventricle was measured by using a lipid peroxidation assay kit (Abcam, Cambridge, UK), as previously described (Loche et al., 2018).

Histology. For histopathological analyses, hearts were fixed for 4 hours in freshly diluted 4% paraformaldehyde at 4° C. and subjected to a graded series of alcohol dehydrations before being embedded in paraffin, and serial 5-mm sections were cut from five mice per group. For immunohistochemical analysis, sections were deparaffinized and rehydrated in graded ethanol concentrations and distilled water. Slides were immersed in diluted, citrate-based antigen unmasking solution (H-3300; Vector Laboratories, Burlingame, Calif.) to unmask antigen using a pressure cooker. Then the slides were treated with 3% hydrogen peroxide prepared in methanol to quench endogenous peroxidase activity. After blocking with phosphate-buffered saline containing 10% goat serum (for rabbit primary antibodies), the sections were incubated with the following primary antibodies overnight at 4° C.: rabbit anti-monocyte-chemoattractant protein 1 (MCP-1) (1:100; 2029, Cell Signaling Technology, Danvers, Mass.), rabbit anti-a-smooth muscle actin (α-SMA) (1:100; 19245, Cell Signaling Technology), and rabbit anti-phosphor-p38 (1:100; 4631, Cell Signaling Technology). After incubation with the primary antibodies, the slides were treated with secondary antibodies supplied in the SuperPicture HRP Polymer Conjugate (87-8963; Life Technologies, Thermo Fisher Scientific, Carlsbad, Calif.). Antibody signals were visualized by peroxidase reaction using 3,30-diaminobenzidine as a chromogen. As a negative control, tissue sections were incubated in the absence of primary antibody to ensure specificity of the primary antibody. At least eight noncontiguous areas from the anterior and posterior portions of each section were photographed for analysis, and three sections from five different mice were measured. For the evaluation of cardiac fibrosis, slides were subjected to Picrosirius red staining (s2365, Poly Scientific R&D Corp, Bay Shore, N.Y.), as previously described (Yuan et al., 2014); images were captured with a Zeiss Axio Z1 inverted microscope. Images were quantified using ImageJ.

Cell Culture. Primary isolated murine adult cardiac fibroblasts, obtained by enzymatic digestion using type II collagenase (Worthington, Lakewood, N.J.) and Protease Type XIV (Millipore Sigma, Burlington, Mass.) according to standard protocols, as described and validated (Kong et al., 2018; Morelli et al., 2019; Wang et al., 2020b), were cultured in Dulbecco's modified Eagle's medium with 10% fetal bovine serum at 5% CO₂ (37° C.), unless otherwise stated.

For the hypoxia assays, fibroblasts were treated with the following medium containing (in mM) 116 NaCl, 26.2 NaHCO₃, 5.4 KCl, 1.8 CaCl₂, 1 NaH2PO₄, 0.8 MgSO₄, 0.01 glycine, and 0.001 (% w/v) phenol red; before the addition to the cells, this medium was saturated for 10 minutes at 1 atm with 95% N₂ and 5% CO₂ mixture. The cells in the described medium were incubated for 6 hours in an anaerobic chamber (hypoxia chamber) filled with the same gas mixture, at 37° C.

Reverse transcription quantitative polymerase chain reaction (RTqPCR) experiments were carried out as previously described and validated (Santulli et al., 2014; Lombardi et al., 2017; Matarese et al., 2020). Briefly, gene expression was determined through means of an AbiPRISM 7300 fast real-time cycler using the power SYBR Green real-time PCR master mix kit and quantified by built-in SYBR Green Analysis; samples were measured in triplicates, and results were confirmed by at least three independent experiments; the relative amount of specific mRNA was normalized to (3-actin.

Chemicals and Reagents. All chemicals were purchased from Millipore Sigma unless otherwise stated. AC261066 was purchased from Bio-Techne Corporation (4046; Minneapolis, Minn.).

Statistical Analysis of Data. All results are presented as means ±S.D. or means ±S.E.M., as indicated in the description of the figures. Statistical analysis was performed using Prism 8.0 software (GraphPad, San Diego, Calif.). All experiments were repeated at least three times. For comparisons of two groups, the unpaired two-tailed t test using (when appropriate) Welch's correction for unequal variances was performed. For comparisons of multiple groups, one-way ANOVA was performed followed by Tukey-Kramer's multiple comparison test. Significant differences were established at a value of P <0.05.

### Results

The RARJ2 Agonist AC261066 Reduces the Post-MI Decline in Cardiac Function and Attenuates Remodeling. Having established that the RARβ₂ agonist AC261066 displays cardioprotective effects by decreasing oxidative stress, it was hypothesized that the antioxidant effects of AC261066 might also mitigate the development of myocardial contractile failure in an in vivo murine model of post-MI heart failure. Accordingly, AC261066 was tested to determine if it could attenuate the decline in LVEF observed post-MI induced by permanent ligation of the LAD coronary artery. Three-month-old wild-type male C57BL/6 mice underwent MI surgery. Mice then remained on their original chow diet and drinking water containing 0.1% DMSO or water containing 3.0 mg AC261066/100 mL in 0.1% DMSO/water for 4 weeks. Ultrasound data were collected at days 0 (baseline), 7, 14, and 23 after permanent LAD ligation. It was found that 23 days after LAD ligation, when an overt heart failure was evident in vehicle-treated mice, the decline in LVEF was significantly less in hearts from mice treated with AC261066 than in vehicle treated mice (Table 1). Moreover, the post-MI decline in left ventricular fractional shortening (LVFS), another measure of left ventricular (LV) contractile function, was significantly smaller in hearts from AC261066-treated mice than in vehicle-treated mice (Table 1). Notably, treatment with AC261066 markedly attenuated post-MI LV remodeling, as proven by measuring the LV end-diastolic internal diameter (Table 1). Furthermore, post-MI lung weight was significantly reduced in drug-treated compared with vehicle-treated mice, strongly suggesting that treatment with AC261066 also decreased pulmonary congestion, another heart failure marker (Table 1). Importantly, there was no difference in troponin I serum levels (measured 24 hours after LAD ligation) between AC261066-treated and vehicle-treated mice, indicating that surgically obtained infarcts did not differ between these two groups. Also, heart rates of drug-treated mice did not change compared with vehicle-treated mice (Table 1). Notably, the diminished decline in contractile function and remodeling was associated with a significant reduction in the staining for MCP-1 [measured by immunohistochemistry (IHC) in sections distant from MI location 4 weeks after surgery] (FIG. 6). Since MCP-1 is a chemokine known to increase in murine MI models and to potentiate LV remodeling, its decrease supports a cardioprotective effect of RARβ₂ activation in the setting of post-MI contractile failure.

TABLE 1
Baseline and post-surgery characteristics
of mice treated with vehicle or AC2610661.

| | Vehicle | | AC261066 | |
|---|---|---|---|---|
| Parameter | Baseline | MI | Baseline | MI |
| HR, bpm | 508 ± 66 | 490 ± 86 | 512 ± 80 | 502 ± 78 |
| HW/BW, mg/g | — | 7.8 ± 0.7 | — | 7.6 ± 0.7 |
| LW/BW, mg/g | — | 7.1 ± 0.6 | — | 6.4 ± 0.5* |
| IVSd, mm | 0.89 ± 0.06 | 0.64 ± 0.04 | 0.87 ± 0.04 | 0.64 ± 0.03 |
| IVSs, mm | 1.23 ± 0.05 | 0.73 ± 0.02 | 2.36 ± 0.05 | 0.75 ± 0.04 |
| LVIDd, mm | 3.37 ± 0.13 | 4.11 ± 0.11 | 3.34 ± 0.18 | 3.82 ± 0.07* |
| LVIDs, mm | 1.99 ± 0.08 | 3.07 ± 0.17 | 1.98 ± 0.16 | 2.7 ± 0.12 |
| LVPWd, mm | 0.86 ± 0.04 | 0.85 ± 0.04 | 0.86 ± 0.04 | 0.9 ± 0.02 |
| LVPWs, mm | 1.05 ± 0.05 | 1.04 ± 0.05 | 1.2 ± 0.06 | 1.17 ± 0.05 |
| LVEF, % | 75.05 ± 2.7 | 43.3 ± 6.4 | 73.69 ± 4.2 | 50.3 ± 4.3* |
| LVFS, % | 40.9 ± 2.3 | 24.8 ± 4.2 | 39.8 ± 3.5 | 29.6 ± 3.1* |
| Serum TnI, ng/mL | — | 60.7 ± 14.2 | — | 61.1 ± 13.9 |

Data are means ± S.D. BW, body weight. HR, heart rate; HW, heart weight; IVSd, end-diastolic interventricular septum; IVSs, end-systolic interventricular septum; LVIDd, end-diastolic left ventricular internal diameter; LVIDs, end-systolic left ventricular internal diameter; LVPWd, end-diastolic left ventricular posterior wall; LVPWs, end-systolic left ventricular posterior wall; LW, lung weight; TnI, Troponin I. Parameters in the MI columns were measured 23 days after the ligation of the left anterior descending coronary artery, except TnI, which was assessed 24 hours after MI, as a marker of the infarct area. n $ 7 mice per group.
*P < 0.05 versus vehicle.

The RARβ₂ Agonist AC261066 Reduces the Post-MI Oxidative Stress in Cardiomyocytes. Since oxidative stress is considered a major cause of ischemic cardiac dysfunction and treatment with AC261066 diminishes oxidative stress, whether the protective effects of AC261066 against the post-MI decline in cardiac contractility and remodeling were accompanied by a reduction in oxidative stress was investigated. For this, the level of MDA, an established marker of oxidative stress, was measured in left ventricles obtained after having euthanized the mice, 4 weeks after surgery. MDA, measured by a lipid peroxidation assay, was increased by ˜70% in the mouse hearts 4 weeks post-MI, compared to the sham group, and that AC261066 treatment reduced MDA levels by ˜60% as compared with untreated post-MI samples (FIG. 7). These results suggest that AC261066 diminished the MI-induced decline in cardiac function and remodeling by attenuating oxidative stress. In agreement with this notion, the increase in phospho-p38 level, another marker of oxidative stress, measured by IHC in sections distant from MI location, was reduced by ˜25% (P <0.01) 4 weeks post-MI in the hearts of mice treated with AC261066 as compared with untreated post-MI samples (FIG. 8).

The RARJ2 Agonist AC261066 Reduces the Post-MI Development of Cardiac Fibrosis. Inasmuch as remodeling is a major consequence of the development of cardiac fibrosis and fibrosis contributes to a long-term disruption of heart function, whether the anti-remodeling effects of AC261066 after LAD ligation were associated with a decrease in interstitial fibrosis was investigated next. For this, collagen deposition was measured in heart sections distant from MI location 4 weeks after permanent LAD coronary artery occlusion. MI induced significant interstitial fibrosis (FIG. 9), measured by Picrosirius red staining. Remarkably, in the hearts from mice treated with AC261066, collagen deposition was reduced by ˜50% (P <0.05) as compared with hearts from vehicle-treated mice (FIG. 9). This finding suggests that AC261066 inhibits post-MI remodeling and contractile dysfunction by limiting excessive fibrogenesis. The expression of α-SMA is recognized to denote the activation of fibroblasts and their transformation into myofibroblasts, another marker of fibrogenesis. Further corroborating that treatment with AC261066 diminished post-MI collagen deposition, the increased expression of α-SMA, measured by IHC, at 4 weeks post-MI in sections remote from MI location, was reduced by ˜50% (P <0.0001) by AC261066 (FIG. 10). These results strengthened the concept that AC261066 reduces the post-MI development of cardiac fibrosis.

The RARβ₂ Agonist AC261066 Increases the Levels of Protective Gene Transcripts in Ischemic Fibroblasts. Since AC261066 decreased collagen deposition and α-SMA expression in post-MI murine hearts, an indication of antifibrotic effects, next tested was whether the antifibrotic effects of AC261066 could possibly derive from a direct protective effect at the fibroblast level. Accordingly, transcript levels were measured by RT-qPCR of three genes, one prooxidant and two protective, in wild-type murine cardiac fibroblasts cultured in normoxic and hypoxic conditions, respectively, in the presence and absence of AC261066. Fibroblasts cultured in hypoxic conditions were found to exhibit an increase in NADPH oxidase 2 (NOX-2) mRNA and decreases in superoxide dismutase 2 (SOD2) and angiopoietin-like 4 (ANGPTL4) mRNAs relative to fibroblasts cultured in normoxia (FIG. 11). The addition of AC261066 (100 nM) did not change the mRNA levels of NOX-2, SOD2, and ANGPTL4 cells in normoxia but significantly reversed the increase in mRNA for NOX-2 and the decreases in SOD2 and ANGPTL4 mRNAs in hypoxic conditions (FIG. 11). NOX-2 is a recognized pro-oxidant enzyme. In contrast, SOD2 diminishes ROS steady-state levels, whereas ANGPTL4 increases nitric oxide levels, decreases collagen expression, and fibroblast to myofibroblast differentiation. Hence, these data suggest that the cardioprotective effects of AC261066 against hypoxia are associated with a direct action at the fibroblast level.

### Discussion

Retinoids are defined as vitamin A (retinol), related metabolites, and synthetic agonists. Interestingly, all-trans retinoic acid has been found to decline in HF, and protective effects in HF have been attributed to it. Here, RARfl, one of the three retinoic acid receptors, was focused on because RARβ plays a role in heart development, and humans with mutations in RARβ show cardiac abnormalities, as well as other features often not compatible with life. Furthermore, RARβ mRNA is highly expressed in both cardiomyocytes and cardio-fibroblasts isolated from adult C57BL/6 mice, and RARβ mRNA expression is detected across cardiomyocyte subtypes and regions.

Having established that a selective RARβ₂ agonist, AC261066, alleviates reperfusion arrhythmias in an ex vivo ischemia/reperfusion model in obese and hypercholesterolemic mice by decreasing oxidative stress, it was hypothesized that the antioxidant effects of AC261066 could also reduce the development of fibrosis in HF. Given that myocardial infarction is a predominant cause of HF and is known to trigger a detrimental fibrotic response, this hypothesis was tested at a systemic, preclinical level in an established clinically relevant murine model of ischemic HF. Ischemic HF is achieved in this model by permanent occlusion of the LAD coronary artery, after which cardiac function is influenced by left ventricular maladaptive remodeling. This model has successfully been used to assess cardioprotective effects of ACE inhibitors, loop diuretics, and antianginal medications.

Oral treatment with AC261066, initiated right after MI surgery and maintained until euthanasia 4 weeks later, was found to reduce the post-MI decline in cardiac function and attenuate remodeling, as indicated by significantly smaller declines in LVEF and LVFS and a concurrent attenuated dilatation of the LV chamber in AC261066-treated mice 23 days after MI surgery, at a time when HF was fully developed. Treatment with AC261066 also resulted in a post-MI decrease in pulmonary congestion, as indicated by a markedly smaller increase in the post-MI lung/body weight ratio. These cardioprotective changes afforded by AC261066 treatment were not confounded by possible surgical artifacts, since infarct sizes were not different in AC261066-treated and vehicle-treated mice, as demonstrated by equal serum troponin I levels measured the first day after surgery.

Having determined that AC261066 alleviates HF by reducing the decline in contractility and remodeling, possible mechanisms of action were searched and genes that are known to be causally associated with the development of HF were measured. Damage from hypoxia-related ROS often precedes structural remodeling of the heart, including fibrosis. Moreover, AC261066 reduces oxidative stress in ex vivo murine hearts subjected to ischemia/reperfusion. Accordingly, our working hypothesis was that AC261066 may attenuate MI-induced cardiac dysfunction by regulating a set of genes that reduce the deleterious effects of ROS and decrease interstitial fibrosis.

We focused first on the possibility that a protective effect of AC261066 against post-MI contractile failure might be associated with intracardiac changes in MCP-1, a cardiomyocyte produced chemokine known to increase in murine MI models and to potentiate detrimental LV remodeling. Oxidative stress increases MCP-1 secretion from cardiomyocytes. Furthermore, MCP-1 null mice, or mice with a deletion of the MCP-1 receptor, have less post-MI LV remodeling, less LV dilatation, and less impairment of LV function. Indeed, in mice treated with AC261066, the diminished decline in contractile function and remodeling was found to be associated with a marked reduction in the staining for MCP-1 in heart sections distant from MI location. This finding supports the concept that a decline in MCP-1 likely mediates, at least in part, the cardioprotective effect of RARβ₂ activation in the setting of post-MI contractile failure.

p38 mitogen-activated protein kinase (MAPK) is a relevant pathway for LV remodeling and dysfunction and post-ischemic HF progression. In fact, oxidative stress leads to increased MAPK phosphorylation in cardiac cells. Moreover, cardiomyocyte-specific activation of p38 MAPK (phosphorylation of p38) in transgenic mice is associated with LV remodeling with interstitial fibrosis, myocyte hypertrophy, and systolic and diastolic dysfunction. Furthermore, p38 MAPK inhibition improves cardiac function and decreases post-MI LV remodeling. Based on these findings, the protective effects of AC261066 observed in post-MI HF, were surmised to involve a reduced p38 MAPK activity. Indeed, these data clearly demonstrate a major reduction in the level of phospho-p38 and number of phospho-p38-positive cells in cardiac sections taken remotely from MI location. This observation strongly suggests that treatment with AC261066 in the setting of MI-induced HF improved LV remodeling and myocardial function by attenuating p38 MAPK activation by oxidative stress. Notably, treatment with AC261066 not only reduced phospho-p38 level but also markedly decreased MDA formation, a characteristic marker of oxidative stress. Thus, treatment with AC261066 is postulated to decrease oxidative stress and, in turn, reduces the activation of p38 MAPK pathway, thereby alleviating LV remodeling and contractile dysfunction in post-MI HF.

Oxidative stress and increased ROS production prevail in the MI setting, and ROS are known to stimulate interstitial fibrogenesis, ultimately leading to maladaptive remodeling and contractile dysfunction. This Example shows that treatment with AC261066 decreases ROS production in post-MI hearts and mitigates remodeling and contractile dysfunction.

Whether the cardioprotective effects of AC261066 involve an antifibrotic step was also investigated. Indeed, treatment with AC261066 markedly decreased collagen deposition, the hallmark of fibrogenesis, in areas remote from the infarct. Fibrogenesis is initiated by fibroblast transformation into active myofibroblasts, exemplified by an increased expression of α-SMA. Treatment with AC261066 diminished post-MI α-SMA expression, further confirming that AC261066 mitigates remodeling and contractile dysfunction in part by diminishing fibroblast activation, thus impeding maladaptive interstitial fibrosis.

As these findings suggested a possible direct effect of AC261066 at the fibroblast level, this hypothesis was tested in cultured murine fibroblasts in normoxic and hypoxic conditions. Accordingly, transcript levels were measured by RT-qPCT of NOX-2, a recognized pro-oxidant enzyme; SOD2, known to diminish ROS steady-state levels; and ANGPTL4, which has been proven to decrease collagen expression and fibroblast activation. Fibroblasts cultured in hypoxic conditions exhibited an increase in NOX-2 and decreases in SOD2 and ANGPTL4 mRNAs relative to fibroblasts cultured in normoxia. Although AC261066 had little effect on normoxic fibroblasts, it partially reversed changes in transcript levels in hypoxic conditions. This data support the notion that the cardioprotective effects of AC261066 in the MI setting are associated in part with a direct action at the fibroblast level. This is a novel finding; although NOX-2 had been implicated in post-MI cardiac remodeling and contractile dysfunction, this action had been attributed solely to an effect on cardiomyocytes. Although the experimental and clinical potential of NOX-2 inhibition in the post-MI setting has been recognized, and diastolic dysfunction and NOX-2 increases have been associated with a loss of RARα signaling, the results here identify for the first time both cardiomyocytes and fibroblasts as targets of the protective role of a selective RARβ₂ agonist in ischemic cardiac dysfunction.

In conclusion, without intending to be bound by theory, it is proposed that AC261066 ultimately alleviates post-MI cardiac dysfunction by sequentially activating RARβ₂ in cardiomyocytes and fibroblasts, then regulating a set of genes responsible for decreasing oxidative stress and ROS production. These AC261066 responsive genes then diminish interstitial fibrogenesis and remodeling. Since MI is a recognized major cause of HF, these data identify RARβ₂ as a pharmacological target in the treatment of contractile dysfunction associated with HF.

It will be appreciated by those skilled in the art that changes could be made to the embodiments described above without departing from the broad inventive concept thereof. It is understood, therefore, that this invention is not limited to the particular embodiments disclosed, but it is intended to cover modifications that are within the spirit and scope of the invention, as defined by the appended claims.

What is claimed is:

1. A method of attenuating or ameliorating cardiac contractile dysfunction in a subject having or having had a myocardial infarction MI or at risk of having a myocardial infarction, comprising administering to the subject a therapeutically effective amount of a selective retinoic acid receptor-β₂ (RARβ₂) agonist or a pharmaceutically acceptable salt, ester, amide, prodrug thereof, or a combination thereof.

2. The method of claim 1, wherein the selective RARβ₂ agonist is AC261066 or AC55649.

3. The method of claim 1, wherein the selective RARβ₂ agonist is administered to the subject during and/or after the myocardial infarction in the subject.

4. The method of claim 1, wherein the selective RARβ₂ agonist is administered to the subject before the myocardial infarction.

5. The method of claim 1, wherein the selective RARβ₂ agonist is administered chronically.

6. The method of claim 1, wherein the selective RARβ₂ agonist is administered acutely.

7. The method of claim 1, wherein the selective RARβ₂ agonist is administered is orally, intravenously, subcutaneously, sublingually, buccally, nasally, intraarterially, intracardiacly, transdermally, transmucosally, intramuscularly, intraperitoneally, topically, or a combination thereof.

8. A method of attenuating or ameliorating cardiac fibrosis in a subject having or having had a myocardial infarction (MI) or at risk of having a myocardial infarction, comprising administering to the subject a therapeutically effective amount of a selective retinoic acid receptor-β₂ (RARβ₂) agonist or a pharmaceutically acceptable salt, ester, amide, prodrug thereof, or a combination thereof.

9. The method of claim 8, wherein the selective RARβ₂ agonist is AC261066 or AC55649.

10. The method of claim 8, wherein the selective RARβ₂ agonist is administered to the subject during and/or after the myocardial infarction in the subject.

11. The method of claim 8, wherein the selective RARβ₂ agonist is administered to the subject before the myocardial infarction.

12. The method of claim 8, wherein the selective RARβ₂ agonist is administered chronically.

13. The method of claim 8, wherein the selective RARβ₂ agonist is administered acutely.

14. The method of claim 8, wherein the selective RARβ₂ agonist is administered is orally, intravenously, subcutaneously, sublingually, buccally, nasally, intraarterially, intracardiacly, intraarticularly, transdermally, transmucosally, intramuscularly, intraperitoneally, ophthalmicly, topically, or a combination thereof.

15. A method of attenuating or ameliorating ventricular remodeling in a subject having or having had a myocardial infarction (MI) or at risk of having a myocardial infarction, comprising administering to the subject a therapeutically effective amount of a selective retinoic acid receptor-β₂ (RARβ₂) agonist or a pharmaceutically acceptable salt, ester, amide, prodrug thereof, or a combination thereof.

16. The method of claim 15, wherein the selective RARβ₂ agonist is AC261066 or AC55649.

17. The method of claim 15, wherein the selective RARβ₂ agonist is administered before, during and/or after a myocardial infarction in the subject.

18. The method of claim 15, wherein the selective RARβ₂ agonist is administered chronically.

19. The method of claim 15, wherein the selective RARβ₂ agonist is administered acutely.

20. The method of claim 15, wherein the selective RARβ₂ agonist is administered is orally, intravenously, subcutaneously, sublingually, buccally, nasally, intraarterially, intracardiacly, intraarticularly, transdermally, transmucosally, intramuscularly, intraperitoneally, ophthalmicly, topically, or a combination thereof.
